(19) United States
(12) Patent Application Publication
(10) Pub. No.: US 2002/0173846 A1
(43) Pub. Date: Nov. 21, 2002

(54) Two part "L"- or "S"-shaped phakic IOL

(76) Inventors: Larry Blake, Coto De Caza, CA (US); Charles D. Kelman, Boca Raton, FL (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 10/056,971

(22) Filed: Jan. 25, 2002

Related U.S. Application Data
(60) Provisional application No. 60/266,394, filed on Feb. 1, 2001.
(60) Provisional application No. 60/269,045, filed on Feb. 15, 2001.

(51) Int. Cl. A61F 2/16

(52) U.S. Cl. 623/6.18; 623/6.43; 623/6.46

(57) ABSTRACT

A two part IOL with a generally “L” or “S” shape but featuring straight or curved “V”-shaped structures which can be inserted through a very small opening by flexing the arms of the “V”-shaped structures up to or over each other is described. This IOL may be used in the anterior or posterior chamber of the eye for phakic or aphakic lenses. After insertion of the haptic into the eye, any type of lens may be attached, especially by use of cleats.

The haptic is a high modulus skeletal frame and the lens is preferably formed of a lower modulus material and is attachable to cleats on the frame.

### FIELD OF THE INVENTION

The present invention generally relates to a two part IOL with a generally “L” or “S” shape but featuring two straight or rounded “V”-shaped structures. More specifically, the present invention relates to an IOL frame which is insertable through an opening as small as 1.0 mm or less by bending the arms of the “V”-shaped structures of the frame together or on top of each other, inserting a lens secondarily into the eye and attaching the lens onto the frame.

### BACKGROUND OF THE INVENTION

The history of intraocular lenses (IOLs) is a long and varied one. Intraocular lenses can be used to treat a wide diversity of eye conditions ranging from cataracts to any type of eyesight correction. In addition, IOLs can be used to replace an irreversibly damaged lens in the eye —aphakic eyes. Alternatively, the lenses can be used in addition to the natural lens to correct the vision —phakic eyes. These lenses can be placed in the anterior or posterior chambers of the eye.

Early IOL researchers were plagued with problems associated with the materials which were obtainable to them at the time (early 1950's) making the lenses too heavy and too large. Surgery of the eye was in its infancy and therefore there were many problems with the surgical procedures. Since that time the quality, size and weight of the optics as well as microsurgical procedures have dramatically improved.

The earliest IOL's were placed in the anterior chamber of the eye, this being the easiest chamber to get to. Along with the early problems with the optics and surgical techniques, placement of a lens in the anterior chamber proved difficult because the anterior chamber is narrow (about 1.5 to 2.5 mm).

The angle between the cornea and the iris was a location within the anterior chamber subsequently used for placement of IOL's. Angle supported anterior chamber IOLs took advantage of the anterior chamber angle to support and fix the IOL in place. By angling the IOL into opposite sides of the anterior chamber, the natural angle was used to keep the IOL from moving. However, early lenses experienced marked problems with endothelial loss due to chafing against the early thick lenses. Later lenses were able to reduce the significance of this problem, but still retained problems associated with placement of the IOL in the chamber angle. The biological properties of that angle make it a very sensitive area. The structures associated with equalizing the internal pressure of the eye are located in that area. Additionally, the tissue in the area is easily irritated and irritation initiates a growth of fibrous tissue, called synechiae. The IOL fixation must be gentle in order to reduce irritation, but stable enough that it will not be easily moveable. This compromise is difficult to obtain. In addition, although the results were excellent in the short-term, there was a significant problem in the long term with altered night vision, loss of endothelial cell populations and alteration of the anterior uvea. These problems as well as the fact that such anteriorly positioned lenses were uncomfortable to the patient, caused many doctors to abandon anterior chamber IOL's.

A third location was developed later and involved implanting a contact lens between the iris and the natural lens. These lenses were called ICL's or implantable contact lenses. However, the ICL's were suspected of initiating cataracts and glaucoma.

As the development of the IOL's became more sophisticated, Ophthalmologists recognized various problems. A typical IOL is composed of an optic, the ‘lens’ part of the structure, and a mounting mechanism called a haptic. The haptics are the part of the IOL that comes in contact with the eye tissue to hold the lens optic in place. There were essentially two major types of haptics which were developed —fiber and plate haptics. Fiber haptics are slender strands of resilient material which are attached at one end to the optic, and which rest, at their other end, against the eye. Fiber haptics have the advantage of being very light and slender. This would seem to make them ideal by causing less damage to the tissue and additionally being aesthetically pleasing because they are very narrow. The slenderness makes it more difficult for someone looking at the patient to see the IOL through the eye. Plate haptics are machined or molded from stock materials and have a central optic and an outer perimeter which rests against the eye. Because of their size, plate haptics tend to be more easily seen from outside in the patient's eye and the addition of extra material weight to the IOL and reduced flexibility as compared to fiber haptics leads to poor fixation and consequent migration or dislocation of the IOL. While fiber haptics have the disadvantage of initiating a process in which the body builds fibrous tissue or synechiae around the fiber haptic which immobilizes the iris, the larger plate haptic very rarely, if ever, causes such a reaction.

The adverse problems associated with the earlier anterior chamber haptic designs encouraged the development of IOL's for the posterior chamber for the majority of implants.

The surgical process may or may not include removal of the diseased natural lens using a process called phakoemulsification. The more standardized procedure for lens implantation involves removal of a diseased natural lens followed by implantation of an artificial lens. Phakoemulsification of the diseased lens is accomplished through about a 2 to 4 mm (small) incision in the eye and through a capsulorhexis incision in the capsule that encloses the lens in the posterior chamber, then an artificial intraocular lens implant is implanted back through the capsulorhexus into the capsular bag. For other types of procedures, the natural lens may not require removal at all.

Related two-piece IOLs of the invention are incorporated herein by reference: U.S. Pat. Nos. 4,056,855; 4,911,715; 5,074,876; 5,769,889; 4,451,938; and U.S. patent application Ser. No. 09/631,576, filed Aug. 4, 2000.

As surgical procedures have developed, there is a trend toward reducing the size of the incision in the eye. Although a 3 mm incision does not usually require sutures for healing, it increases the chances of astigmatism or infection, heals slower, and may provide for a slower operation than if an incision of less than 3 mm is used. However, presently IOLs cannot be inserted into a very small incision, as small as about 1 mm.

### SUMMARY OF THE INVENTION

Accordingly, an intraocular lens (IOL) has been developed. The intraocular lens features an optic and a haptic. The haptic is generally “L”- or “S”-shaped, but features straight or curved “V”-shaped structures which are relatively rigid, because they are fabricated from higher modulus (harder) materials, but, they are very narrow so they are flexibly springy when thin. This permits the arms of the haptic to be bent close to, or over each other to fit through a small incision. The mixture of the general “L”-shape with “V”-shaped elements of the haptic allows insertion of the haptic through an opening in the eye as small as about 1 mm. The haptic also features a fastening structure for the separate foldable optic, preferably a cleat. The foldable optic is inserted into the eye through the same ultra small incision and attached to the haptic, preferably the haptic cleat, by way of one or more formed apertures or eyelets on the optic. The arms which are typically made up of a frame lens support member and haptic support member can bend or flex together such that the frame can be manipulated through an incision less than about 2 mm and down to about 1 mm and potentially as narrow as about 0.5 mm with minimal contact with the tissues.

The narrow shape of the haptic arms of the preferred embodiment allows for very low forces when flexed, reducing perceptible sensitivity or irritating trauma. Disc shaped support feet on the frame are similar to older designs of plate lenses and minimize discomfort or synechiae.

The higher modulus springy polymeric material may be selected from polyimide, polyetheretherketone, polycarbonate, polymethylpentene, polymethyl methacrylate, polypropylene, polyvinylidene fluoride, polysulfone, polyphenylsulfone and polyether sulfone. Preferably, the higher modulus material is polymethylmethacrylate (PMMA). Preferably, the higher modulus material has a modulus of elasticity of about 100,000 to about 500,000 psi, even more preferably about 450,000 psi and has a Rockwell M scale hardness of 90 to 95.

In one embodiment, the haptic comprises a two point frame. The two point frame has two contact areas or zones which contact the tissue of the eye. In a further embodiment, the haptic comprises a three point frame. The three point frame has three contact areas or zones which contact the tissue of the eye. The frame forms three feet which may be fabricated from a single uniform piece of material. The haptic may contain a cleat for attachment of the lens.

In a further embodiment, the haptic comprises a four point frame. The frame forms four feet which may be fabricated from a single uniform piece of material. The haptic may contain a cleat for attachment of the lens.

The optic may be any type of lens. Preferably, the optic is a refractive lens, or an interference lens, producing a thin optic. The optic could be toric, aspheric, multi-element, positive or negative.

### BRIEF DESCRIPTION OF THE DRAWINGS

FIG. 1 is a simplified representation of the cross-sectional physiology of the eye with an IOL in accordance with the preferred embodiment implanted in the anterior chamber.

FIG. 2 is a plan view of the multi-part IOL in accordance with the preferred embodiment of the three-point haptic, showing the flexibility of the “V”-shaped structures as well as the preload.

FIGS. 3A and 3B are alternate embodiments of the eyelet and cleat attachment of the haptic and the lens.

FIG. 3C is a perspective view of an embodiment of the eyelet and cleat attachment of the IOL and the slender optic showing that the eyelet can be fabricated separately from the lens using a monofilament fiber.

FIGS. 3D is a perspective views of an embodiment of the cleat which is machined separately from the haptic and then attached onto the haptic.

FIGS. 3E and F are perspective views of the cleat which shows that it can be machined separately and attached.

FIG. 4 is a plan view of the multi-part IOL in accordance with the preferred embodiment of the four-point haptic, showing the flexibility of the “V”-shaped structures.

FIG. 5 is a plan view of an alternative embodiment of the three-point haptic in which the lens support members are separate from the flexing support members.

FIG. 6 is a plan view of an alternative “V” shaped structure for the haptic of the invention.

FIG. 7 is a side view of the haptic as it is being manufactured from the blank in accordance with the preferred embodiment.

FIGS. 8A-D are plan views of the method of making the haptic of the preferred embodiment.

FIGS. 9A-E are plan views of the haptic being inserted into an eye through an ultra-small incision. The arrows indicate which way the haptic is moved to allow insertion.

FIGS. 10A-C are plan views of the IOL of the preferred embodiment being surgically assembled in the eye.

FIG. 11 is an isometric view of the final packaged product of the preferred embodiment.

FIGS. 12 A-H are plan views of the surgery which introduces the multi-part IOL of the preferred embodiment into the eye.

FIGS. 13A-C are plan views of the haptic of the preferred embodiment, showing the rounding of the haptics to produce more space for attachment of the eyelet to the cleat.

FIG. 14 is a simplified representation of the cross-sectional physiology of the eye with an IOL in accordance with the preferred embodiment implanted in the posterior chamber.

FIGS. 15A-C are plan views showing three embodiments of the haptic of the multi-part IOL in accordance with the preferred embodiment of the “S” shaped haptic.

FIG. 16A is a side view of a further embodiment of the “S” shaped haptic. FIGS. 16B and 16C are closed and open versions of the “S”-shaped haptic.

FIGS. 17A-C are plan views of the haptic of FIG. 16C with the optic attached. FIGS. 17A and C show that the lens can be placed above or below the cleat to place the optic within the vault or outside the vault. FIG. 17B is a side view of the haptic and optic of FIG. 17A.

FIGS. 18A-E are plan views of the embodiment shown in FIG. 16B being inserted into the eye.

FIG. 19 is a plan view of the embodiment shown in FIG. 16B showing the flexibility of the closed haptic as it is being inserted into the eye.

FIGS. 20A and B are plan views of the embodiment shown in FIG. 16C showing the flexibility of the rounded “V”-shaped structures as they are being inserted into the eye.

### DETAILED DESCRIPTION OF THE PREFERRED EMBODIMENT

Accordingly, a haptic with a generally “L”, plural “L” or “S” shape but featuring one or more straight or curved (rounded) “V”-shaped structures has been developed for a two part IOL. This frame with narrow haptic structures is insertable through an opening in the eye as small as about 1 mm by a combination of manipulating the frame into the incision and flexing the arms of each “V”-shaped structure of the frame together or on top of each other. The IOL further comprises a lens which can then be implanted in the eye. This frame haptic is also lightweight, springy and non-irritating, low cost, surgically implantable with a minimum of trauma to the eye, aesthetically pleasing, and does not support fibrous tissue growth. This IOL works in the anterior or posterior chamber of the eye for phakic or aphakic lenses. This haptic additionally comprises a fastener for a separate optic.

This generally “L” or “S”-shaped IOL frame is a haptic system based on a high modulus, shaped skeletal frame or plate haptic. The more rigid material frame or haptic ensures that the lens and springy haptic assembly also exhibits high elastic memory, will maintain its shape, and will stay ideally situated in the anterior chamber angle of the eye or in the posterior chamber. In contrast, a haptic of a single soft material will not maintain a desirable shape and will be more noodle-like in its spirit and will not be stable in the eye. The springy skeletal frame segments of the preferred design are thicker axially than they are radially which will minimize vaulting (i.e. axial motion) due to normal movements of the eye. Additionally, the eyelet aperture is slightly larger than the cleat, allowing nominal frame flexure without effecting the optic.

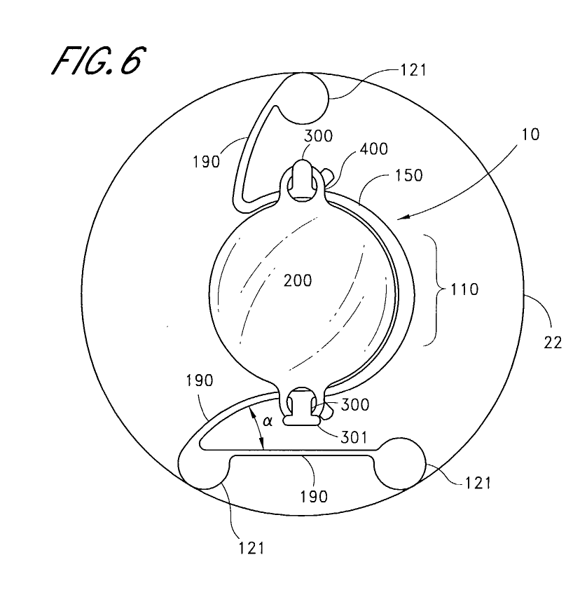

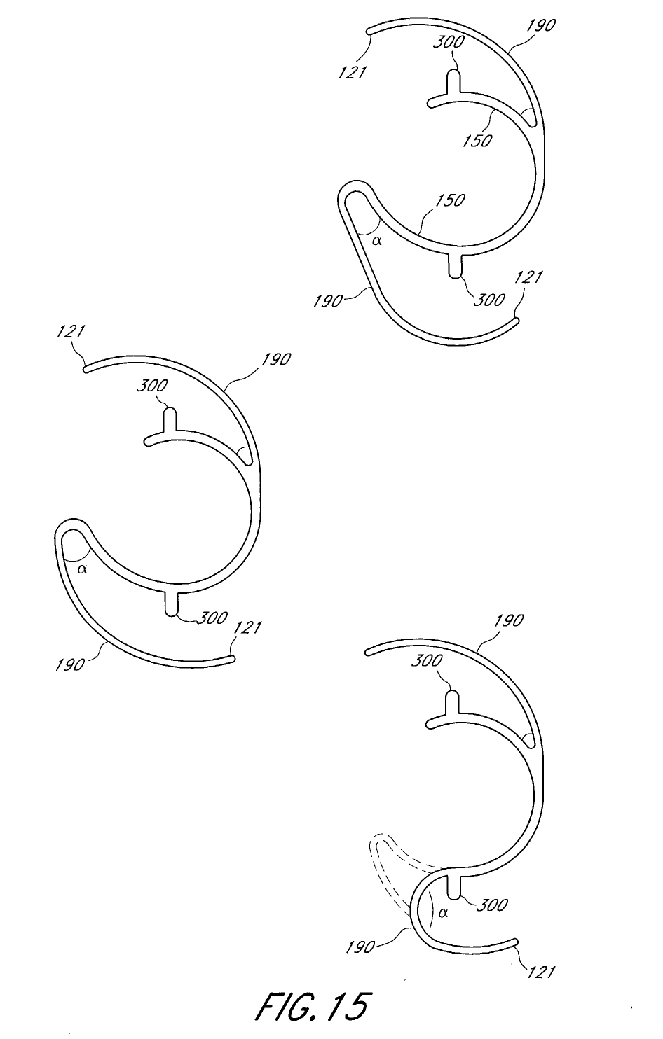

The embodiment shown in FIG. 5 and related embodiments with additional “V”-shaped structures typically allow three contact points within the eye chamber and are advantageously found to work optimally within the anterior chamber. The embodiment shown in FIG. 15, having more curved “V”-shaped structures typically allows for two contacts within the eye and was advantageously found to work optimally within the posterior chamber, particularly for aphakic eyes. A further embodiment is envisioned which contains a circular optic support. It is envisioned that any of the embodiments shown herein may be used within any chamber for phakic or aphakic eyes. In addition, the multi-part IOL allows for replacement of either part when necessary with little or no trauma to the eye. For example, if the patient's refraction changes, the lens may be replaced without removing the haptic. Alternatively, if the haptic is found to be a less than optimal size, the haptic may be removed without removal of the lens. Additionally, the haptic could be easily re-positioned within the eye or removed and replaced in a different position in the eye.

The ability to replace the lens makes the IOL advantageous for use in any patient who may experience refractive changes in the eye. For example, in children, when a lens is implanted, it may need to be changed every 2 years or more when the eye changes refraction. However, with the embodiments shown herein, the optic may easily be removed and replaced with the correct lens. This allows a continuous adjustment for perfect vision.

The IOL described herein may be inserted into the eye through a very small opening of less than 2.5 mm and as little as 1.0 mm or less. This is difficult to do, since many IOLs may experience damage when inserted into a small opening. In addition, this allows for quicker healing and recovery and reduces the chances of infection.

Insertion of the lens into the eye involves a technique which snakes or manipulates the haptic into the eye with or without flexure of the haptics. The optic may be inserted separately and the two pieces may then be assembled within the eye. Alternatively, the pieces may be partially or completely assembled outside of the eye and inserted together. For example, the haptic may be inserted up to the furthest cleat. The optic may then be partially assembled by attaching the eyelet of the optic onto the cleat of the haptic. The assembly may then be completed within the eye or as the partially assembled IOL is inserted. Alternatively, the optic may be placed within the eye and the haptic inserted after. Assembly may then take place within the eye. The need to insert a haptic after the optic may arise due to damage or miss-sizing of the original haptic. Once the original haptic is removed, the correctly sized or undamaged haptic may be inserted.

The surgery which involves implanting the IOL of the preferred embodiments into the eye is preferably as brief as possible to provide for the least discomfort to the patient, the fastest healing time (due to less trauma) and the least risk of infection. Thus, after the incision is made and the viscoelastic inserted, implanting the frame should take no more than about two minutes, preferably 1 minute, more preferably 45 seconds, 30 seconds or even more preferably no more than about 15 seconds.

After insertion of the lens into the eye, assembly of the first eyelet onto the frame should take no more than 2 minutes, preferably 1 minute, more preferably 45 seconds, 30 seconds or even more preferably no more than about 15 seconds. Similarly, the second cleat assembly should take no more than 2 minutes, preferably 1 minute, more preferably 45 seconds, 30 seconds or even more preferably no more than about 15 seconds. Any surgery and implant assembly in the living body that involves more complex designs including screws or threads or hinge mechanisms or even banding would probably exceed these times.

The intraocular lens may be used to correct any malfunction of the eye which involves the lens, including myopia of from −8 to -20 D, cataracts, phakic or aphakic eyes, hyperopea, prespyopia, or any requirement from about -20.0D to about +30.0D.

Dimensions for the haptics of the preferred embodiments may be varied so as to fit the patient's eyes. Typically, the haptic may be from about 11 to 15 mm to fit the anterior chamber, including 11.5 mm, 12 mm, 12.5 mm, 13 mm, 13.5 mm, 14 mm, and 14.5 mm as well as increments between these dimensions, preferably from about 12 to about 13.5 mm. Typically, the haptic may be from about 11 to 15 mm to fit the posterior chamber, including 11.5 mm, 12 mm, 12.5 mm, 13 mm, 13.5 mm, 14 mm, and 14.5 mm as well as increments between these dimensions, preferably from about 12.5 to about3.0 mm.

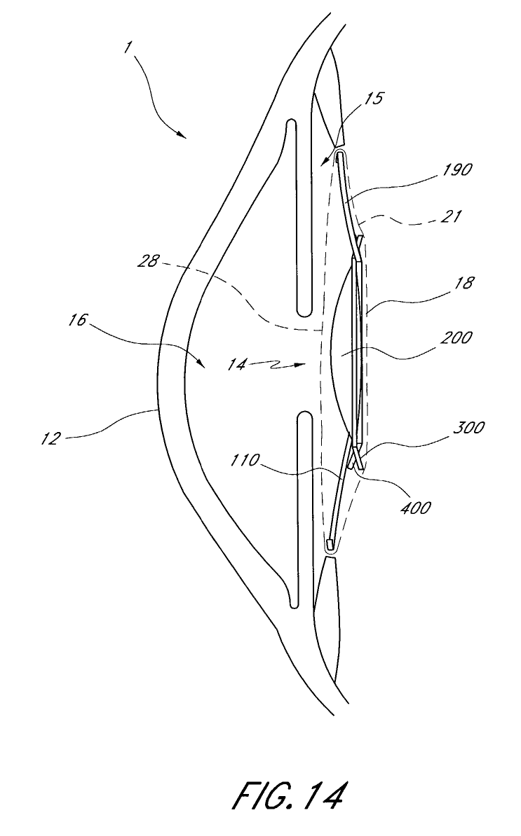

Other traits which are advantageous for a posterior chamber haptic include the haptic frame angling or vaulting backward instead of forward. The vaulting allows for a safer fit within the eye, reduces the possibility of the lens touching the tissue of the eye and reduces the chances that the haptic feet will obstruct the eyelet/cleat attachment. The vault 180, as shown in FIG. 14 could be from about 0.02 mm to about 1.0 mm as required for the patient, including 0.05, 0.1, 0.2, 0.3, 0.4, 0.5, 0.6, 0.7, 0.8, and 0.9 mm.

The haptics may be the same width and thickness or may vary within parameters. Typical widths and thicknesses for the haptics include from about 0.05 to about 0.50 mm, including about 0.05, 0.75, 0.9, 0.1, 0.12, 0.13, 0.14, 0.15, 0.16, 0.17, 01.8, 0.19, 0.2, 0.21, 0.22, 0.23, 0.24, 0.25, 0.26, 0.27, 0.28, 0.29, 0.30, 0.31, 0.32, 0.33, 0.35, 0.38, 0.40, 0.41, 0.42, 0.45, 0.47, 0.48, and 0.49 mm.

The cleat and eyelet attachment of the preferred embodiment provides for ease in attaching the lens to the haptic. There are two features of the haptic that cause the bowing out of the haptic, the vaulting, which is produced during machining of the haptic, and the preload, which enhances the vaulting. These features allow the haptic to provide for slight variations in the corneal angle and physiology and movement of the eye. In addition, the vaulting of the haptic allows the surgeon to more easily attach the lens to the haptic with little or no obstruction. For this reason, the axial spaces on the haptic may preferably be not less than the eyelet thicknesses (or the vault space). This is because, to attach the lens to the haptic, the surgeon may wish to slide the lens body with the eyelet over the frame, through the frame and then to retract the lens in reverse so the eyelet can pass along and under the frame and between the frame and the iris tissue. For the subsequent hooking there will be an additional step of stretching and/or compressing (flexing) the frame up to about 0.5 mm.

The embodiments of the multi-part IOLs will now be described with reference to the Figures and Examples.

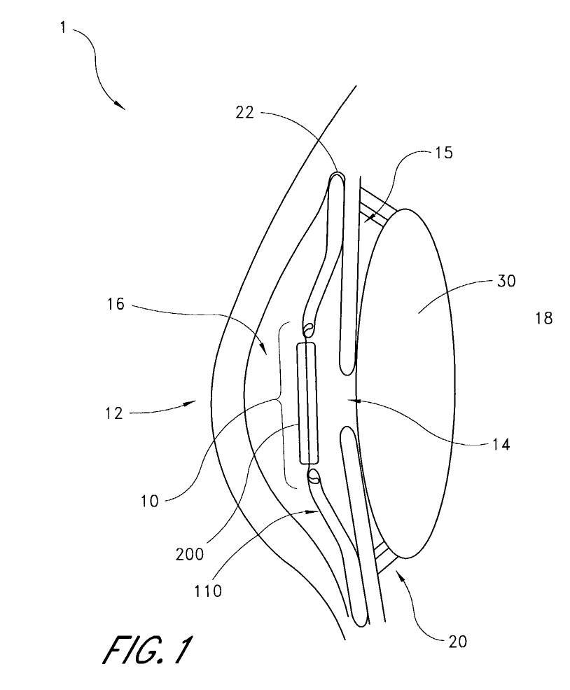

Referring to FIG. 1, the cornea 12 serves as a refracting medium in addition to its function as the anterior wall of the eye 1. The pupil 14 and the iris 15 of variable aperture are located behind the cornea 12 and divide the eye 1 into an anterior chamber 16 and a posterior chamber 18. The natural crystalline lens 30 is connected by zonular fibers to a peripheral muscle about the lens 30 known as the ciliary muscle 20. The lens may be positioned in the anterior angle 22.

The more standardized procedure for the removal of a diseased natural lens 30 followed by implantation of an artificial lens involves the phakoemulsification of the diseased lens through a small incision in the eye and through a capsulorhexis incision in the capsule that encloses the lens in the posterior chamber 18, then an artificial intraocular lens implant is implanted back through the capsulorhexus into the capsular bag. For other types of procedures, the natural lens 30 may not require removal at all. The IOL 10 of the preferred embodiment includes a separate centrally located optical zone or lens 200 and may be configured for implantation into either the anterior 16 or posterior chamber 18 and may be used for either procedure set out above. The haptic 110 of the IOL 10 extends radially outwardly in the general plane of the optic 200.

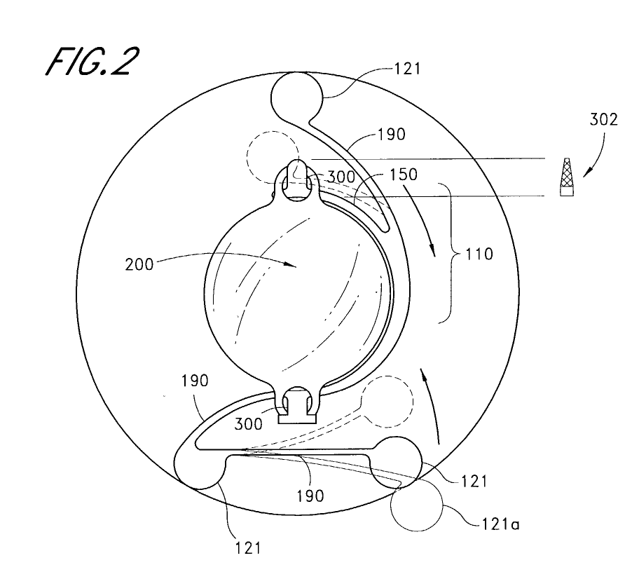

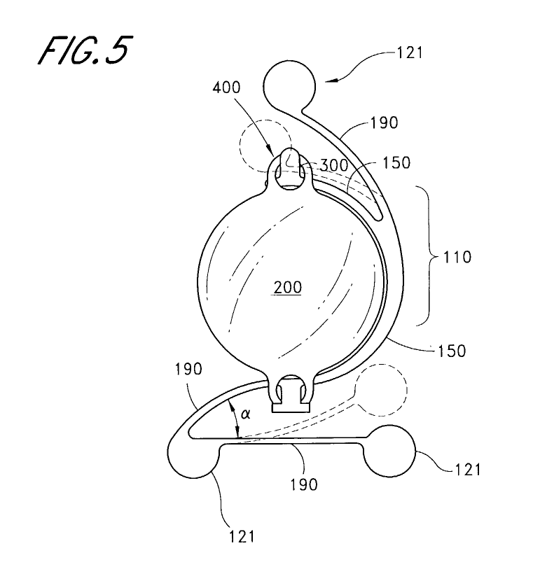

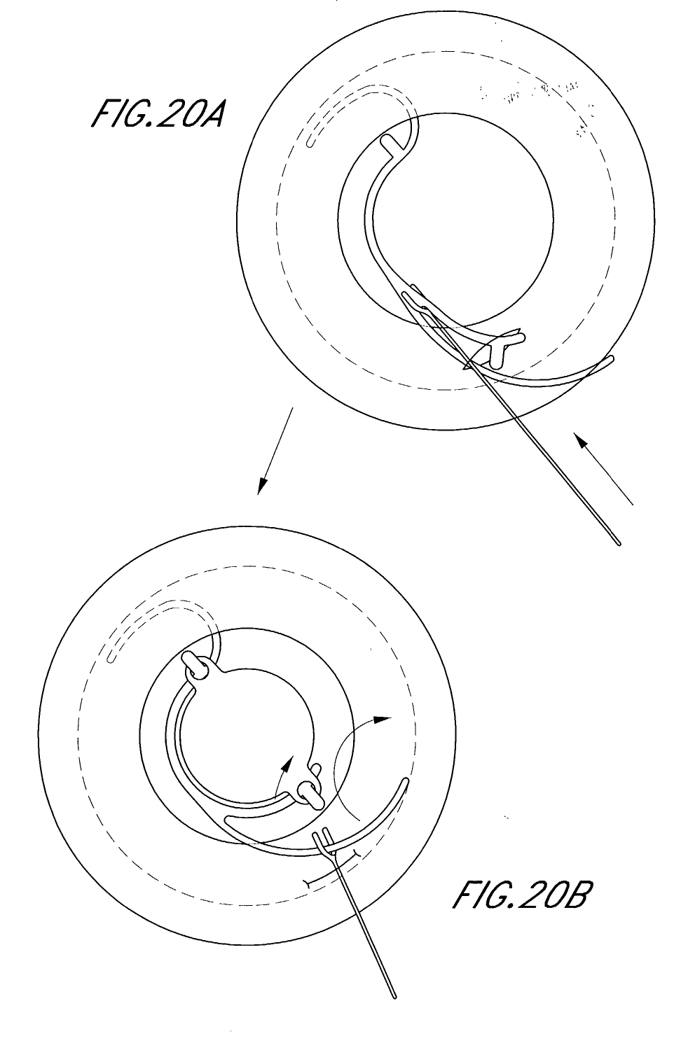

With reference now to FIG. 2, the separable plural-part IOL arranged and configured in accordance with certain features, aspects and advantages of the present invention will be described in detail. FIG. 2 is a plan view of the thin frame haptic of a plural part IOL 10 in accordance with the preferred embodiment. The intraocular lens 10 is generally comprised of a lens optic 200 and a lens skeletal frame haptic 110. The thin frame haptic 110 includes at least three feet 121 and plural flexible support members 190. The thin frame/haptic 110 in FIG. 2 comprises at least three areas which come in contact with the eye tissue. The feet 121 function like plate haptics and, as such, differ from the fiber haptics of the prior art. The three feet 121 and multiple flexible support members 190 are arranged in an approximate forward or backward “L”-shape with at least one “V”-shape. By “V” shape, it is envisioned that there is at least one “corner” or “angle” alpha (α) which is as great as 90° or less, but preferably from about 15 to 50°, more preferably between 30 and 45° (angular degrees). In addition, there is at least one lens mounting member 150 which is structurally immobilized and produces, when paired with a springy, flexible support member 190, a “V”-shaped structure. It is envisioned that the flexible support member 190 can function as a lens mounting member 150, allowing the lens 200 to be attached directly to the flexible support member 190. The combination of the flexible support member 190 with the lens mounting member 150, produces a “V”-shaped structure, the arms of which can be flexed during insertion through an incision in the eye 1. The arms of the “V” shaped structure can include one flexible support member 190 and one lens mounting member 150, two flexible support members 190 or mixtures of the two (see the alternative embodiment of FIGS. 4-6 described below). The flexible “V” shaped structure allows the haptic 110 to be inserted into a very small incision by bending the haptic elements (or arms) and, more specifically (see FIG. 20A), by bending the flexible support member 190 of the “V”-shaped structure, up to or over, the structurally immobilized lens mounting member 150. The feet 121 can be from about 0.5 to 2 mm in diameter, but preferably about 1 to 2 mm in diameter. The very small incision is preferably less than 3 mm, more preferably less than 2 mm, and even more preferably less than 1.5 mm and most preferably less than about 1.0 mm. The maximum dimension of each section along the length of the haptic 110, when bent, is less than the incision. The haptic can be temporarily bent to about 1 to 1.5 mm or up to 3 mm as the frame is passed through the incision. It is understood that, due to the fact that living tissue is elastic and will yield a little, the incision in the eye will stretch a small amount without damage to the tissue. For example, it has been observed that a 2.5 mm incision will stretch to as much as 3 mm, to allow passage of a 3 mm wide haptic arm. The “V”-shaped structures of the preferred embodiment are compliant enough that the living tissue will further urge the bending of the “V”-shaped structures as they are passed through the incision. Therefore, the minimum size of the incision is about equal to the diameter of the foot.

FIG. 2 illustrates a preferred embodiment of the trailing haptic which is offset from the nominal haptic diameter as much as about 1 mm, preferably about 0.3 to 0.6 mm, alternatively about 0.30 mm to 0.50 mm, and as low as 0.1 mm, including 0.2mm, 0.25 mm, 0.35 mm, 0.45 mm, 0.55 mm, 0.65 mm, 0.7mm, 0.75mm, 0.80 mm, 0.85mm, 0.90 mm, and 0.95 mm. This will result in a slight preload on the haptic when positioned within the eye. For example, the diameter of a typical eye angle is between about 12 mm to 14 mm. In the preferred embodiment, one foot 121a is manufactured to extend beyond this by up to about 1 mm so that when it is placed in the eye, there is a preload on the frame reducing the tendency for “lifting up” of the frame. In this figure it can be seen that the diameter of the haptic is up to 1 mm greater than the diameter of the eye, which causes a preload.

With reference to FIG. 2, the thin frame haptics 110 and feet 121 are preferably manufactured from a high modulus material. High modulus materials are generally relatively stiff, or hard, but springy and permit relatively little elongation before they break and in addition they exhibit high memory. Such materials are often brittle and have a high permanent set, but retain their shape after formation. Preferably, the high modulus material is a biocompatible thermoplastic such as polyimide, polyetheretherketone, polycarbonate, polymethylpentene, polymethylmethacrylate (PMMA), polypropylene, polyvinylidene fluoride, polysulfone, and polyether or polyphenyl sulfone or polyester types such as dicyclopentadiene, know as Zeomex. These are often referred to as “engineering plastics”. They have high tensile strength and are biocompatible, hydrolytically stable, and some are autoclavable for sterility, and have a high modulus ranging from a tensile modulus of about 100,000 to 500,000 psi (using test method D 638 of the ASTM). The material can be clear, opaque, or tinted, but is preferably clear. However, in many cases, even a tinted material, if produced thinly enough, will appear clear in the eye.

With further reference to FIG. 2, the separate lens optic 200 can be any type of lens, elastomeric or polymeric optical material. The optic 200 can be any type of optic known to the skilled artisan, including a simple refractive lens, a monofocal lens, a toric lens, a bifocal lens, an interference lens, a positive lens, a negative lens, an aspheric lens, a standard power monofocal lens, a multi-focal spheric lens, a multiple optic lens, an interference lens, a thin lens (film-type technology), a radially or laterally non-symmetrical lens or a defocusing (presbyoptical) lens. The lens may be hydrophillic or hydrophobic. Dimensions of the optic may be from about 2 to about 7 mm, including 2.5 mm, 3.0 mm, 3.5 mm, 4.0 mm, 4.5mm, 5.Omm, 5.5mm, 6.0 mm, and 6.5 mm or increments between these dimensions. Preferably, the optic is from about 5.0 to about 6.0 mm. The thickness of the optic may be from about 0.05 to about 1 mm thick, including 0.1, 0.15, 0.2, 0.25, 0.3, 0.35, 0.4, 0.45, 0.5, 0.55, 0.6, 0.65, 0.7, 0.75, 0.8, 0.85, 0.9, and 0.95mm or up to 1.5mm. The lens can be made thinner by using the polychromatic diffractive lens disclosed in U.S. Pat. No. 5,589,982 which is incorporated herein by reference. Optionally a regular lens can be made thinner by edge-bonding, or bonding the haptic to the outside of the lens as disclosed herein rather then burrowing a hole into the side of the lens as is done routinely. The lens optic 200 can be made of silicone (Optical index N=1.40 to 1.46), soft acrylic (N=1.40 to 1.46), hydrophilic acrylic, or methyl methacrylate (N=1.49) or polyphenylsulfone (N=1.67) or any acceptable optical material. Alternatively, the lens optic 200 may be made of the same material as the thin frame haptic 110 and can be made of a material having a hardness as low as 15 shore on the A scale.

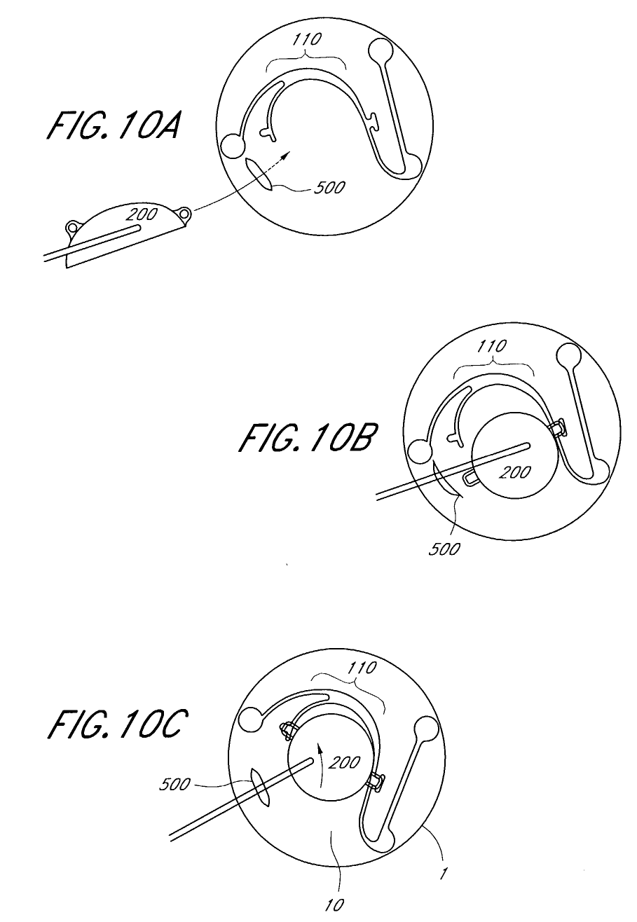

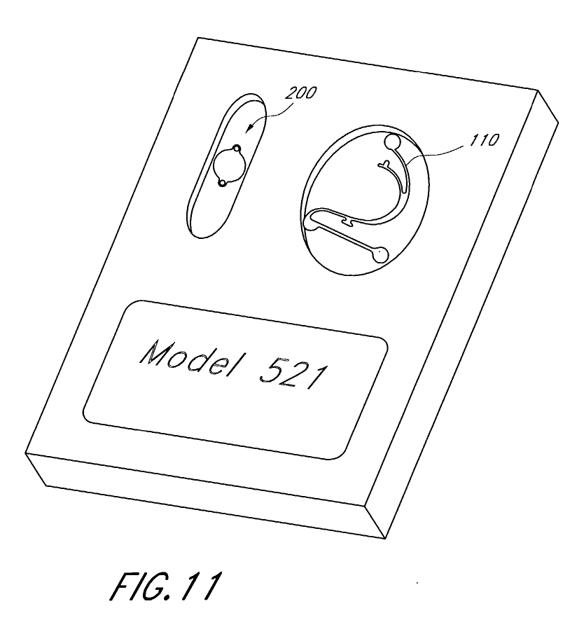

The lens optic 200 can be attached to the frame haptic 110 in a variety of ways. A preferred embodiment is shown in FIG. 2, in which the optic includes eyelets 400 which permit attachment of the lens 200 to a pair of cleats 300 on the haptic. It is envisioned that the surgeon can attach the optic 200 to the haptic 110 within the eye 1 using a forceps or other instrument. The haptic 110 is inserted into the very small opening and positioned in the eye as desired (see FIGS. 9A-E, explained below). Then the optic 200 is rolled or folded as needed and inserted into the eye with forceps and attached to the furthest cleat 300 from the opening (see FIGS. 10A-C, explained below). As the forceps are removed, the eyelet 400 on the other side of the optic 200 can be attached to the cleat 300 closest to the opening. In a preferred embodiment, the optic 200 is produced of a material with a lower modulus then the haptic 110, thus allowing the eyelet 400 to be slightly stretched as the haptic 110 is slightly sprung to allow a stronger attachment of the optic eyelets 400 to the cleats 300 on the haptic 110. The optics of this invention can be made with very thin edges (as thin or as low as 10 μ) to help reduce edge glare.

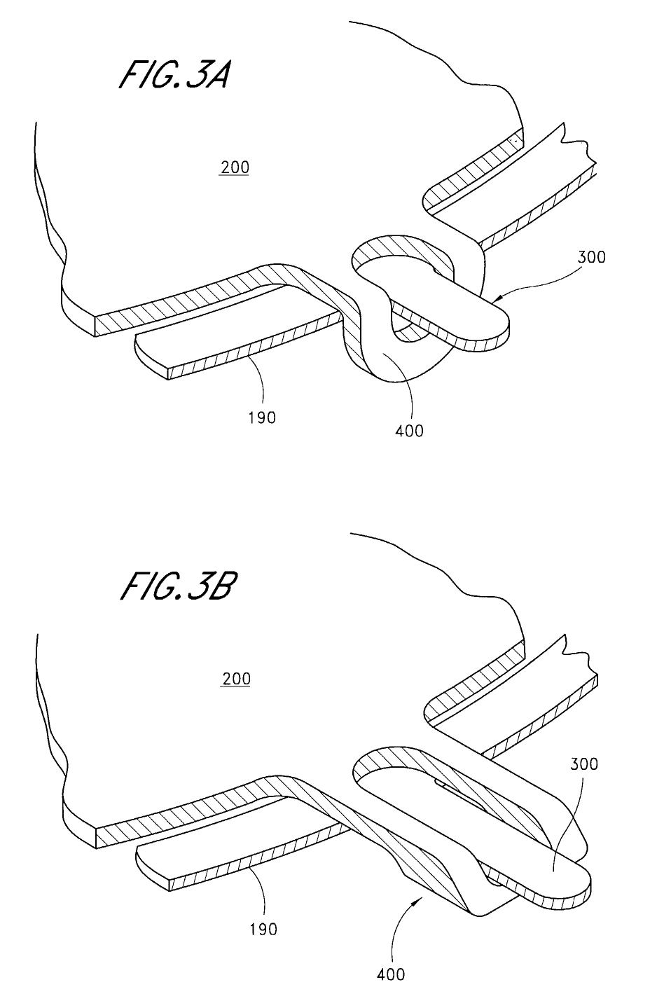

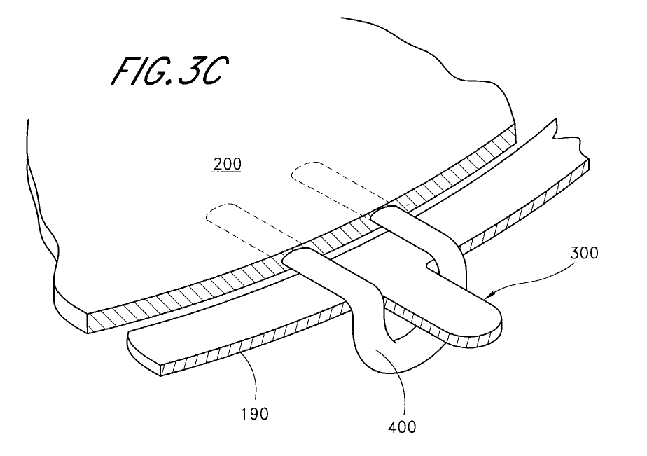

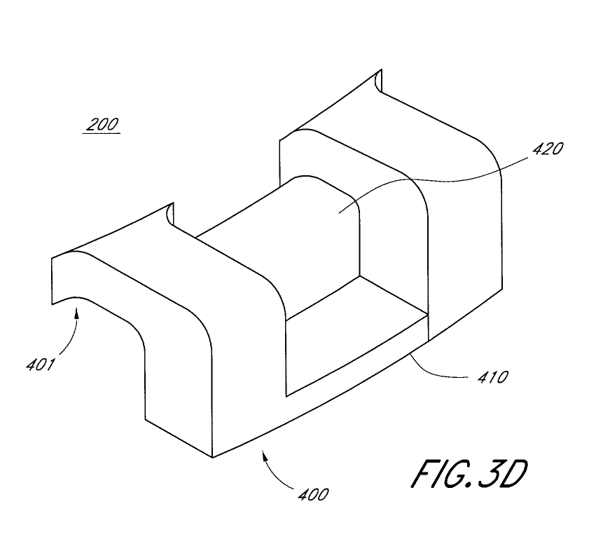

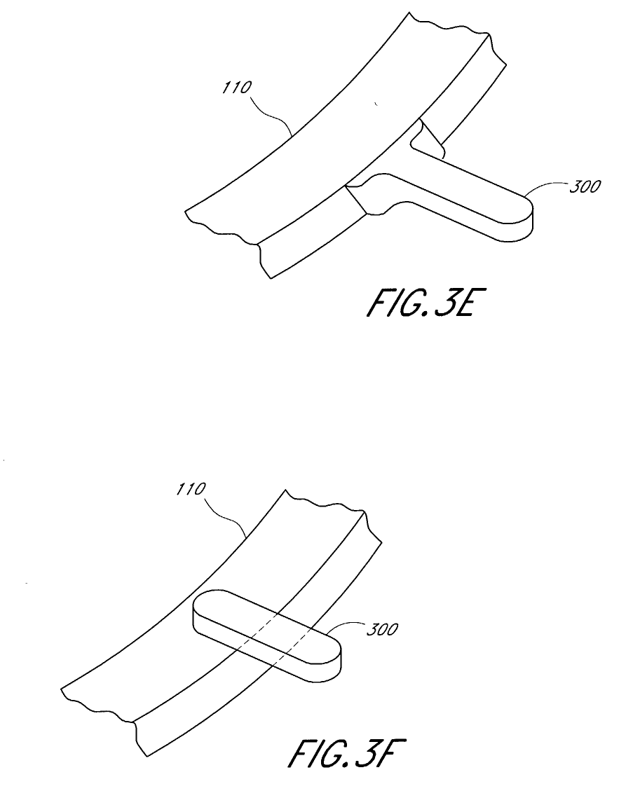

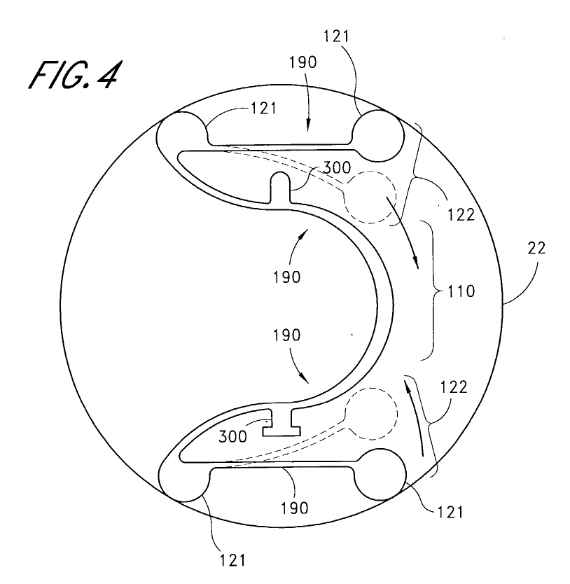

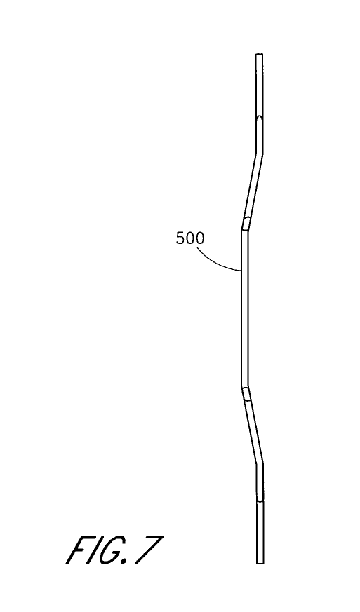

With reference to FIGS. 2 and 3, it can be seen that the cleats 300 may be formed in a variety of shapes. The cleat 300 may be smooth or may have prongs 301 (see FIG. 6). An advantage of the prongs 301 is that the lens 200 will be less likely to become detached from the frame 110. In addition, a pronged cleat 301 will help to keep the first eyelet 400 in place as the second eyelet 400 is being manipulated during attachment of the lens 200. The cleats 300 may be arranged symmetrically, alternatively, the cleats 300 may be arranged so that they are not diametrically opposed. An advantage of this is that lenses 200 can be used which are not symmetrical, allowing for treatment of astigmatism. In addition, excess cleats 300 may be added to allow for repositioning the lens 200 by hooking it onto different cleats 300. For example, the cleats 300 may be arranged in multiple equal angle increments so the optic can be unhooked and rotationally adjusted to a different position. Thus, if a lens 200 needs to be inserted and positioned in a specific orientation, it can be more easily done with this asymmetry as a visual aid. In addition, multifocal optics 200 can be used which allow for correction of a variety of eyesight imperfections. The addition of a third cleat 300 would allow control of asymmetric as well as symmetric features. The cleats 300 may be machined as part of the IOL, and in this case the haptic 110. Alternatively, as shown in FIGS. 3E and 3F, the cleats 300 may be machined separately and attached to the haptic 110. Attachment may be by any method known to one of skill in the art, but may include, adhesive solvent sonic, laser or other thermal or fusion processes. Separate machining and placement of the cleats 300 may allow for tinting in order to make them more identifiable to the surgeon during surgery.

In one embodiment, shown in FIGS. 3A though 3D, the eyelet 400 is shaped in such a way that the cleat 300 will not easily be detached. FIGS. 3A and 3B show those embodiments of the cleats 300 and the eyelets 400 of the invention which have been shown to work particularly well for the intended purpose. FIGS. 3C and 3D provide further embodiments of the eyelet 400. With reference to FIG. 3D, eyelets 400 include apertures 420 which may be about 0.25 to 2.0 mm in diameter, including 0.5, 0.75, 0.76, 0.8, 0.9, 1.0, 1.2, 1.4, 1.5, 1.6, 1.7, 1.8, and 1.9 mm. The thickness of the eyelet 400 may vary, but is typically between about 0.25 to 1.0 mm, including 0.33, 0.38, 0.4, 0.5, 0.6, 0.7, 0.8, and 0.9 mm. The cross bar 410 may be the same width or in some embodiments may be thinner. Preferably, the cross bar 410 is from about 0.02 to about 0.35 mm, including 0.05, 0.075, 0.1, 0.125, 0.15, 0.175, 0.2, 0.225, 0.25, 0.275, 0.3, and 0.325 mm. The eyelet 400 may include a drop 401 of between about 0.15 to 1.0 mm, including 0.3, 0.4, 0.6, 0.7, 0.8, and 0.9 mm. The eyelet 400 may be any shape, preferably rectangular, circular or oval and the bars (including the crossbar 410) of the eyelet 400 may be round, square, or rectangular and may additionally comprise rounded, tapered or sharp corners. The eyelet aperture 420 may be 0.10 to 0.35 mm larger in diameter than the cleat 300 post width to allow for frame 110 movements without imposing stress to the optic 200. This allows for clearer vision during normal movements of the eye 1. The eyelets 400 may be angled or offset to help assembly and to assure that the cleats 300 will remain attached within the eye 1 and during assembly of the IOL 10. The cleats 300 are preferably pointed or beveled 5° or up to 15° thinner at the tip (See 302 in FIG. 2). This will help guide the eyelet 400 of the lens 200 onto the cleat 300 of the haptic 110. The cleats 300 may be formed as part of the haptic 110 or may be produced of a different material and attached by a variety of bonding techniques known to one of skill in the art. The eyelets 400 may be machined as part of the lens 200 or haptic 110 or may be machined separately. The eyelet 400 and cleat 300 attachment may be as shown in FIG. 2 with the eyelet 400 on or as part of the lens 200 and the cleat 300 on or as part of the haptic 110. Alternatively, the eyelet 400 may be on or part of the haptic 110 and the cleat may be on or part of the lens 200. In FIG. 3C an embodiment of the eyelet 400 is shown which is machined separately and attached adhesively or mechanically hooked into the lens 200. The eyelet 400 may be attached similarly in any way known to one of skill in the art. The embodiment of the eyelet 400 shown in FIG. 3C is a filament eyelet 400 having as much as, or less than, the thickness of the lens 200 that it is attached to. If machined separately, the eyelet 400 can more easily be tinted opaque so as to be seen more easily during surgery.

Therefore it is envisioned that the cleats 300 could be used to attach any type of IOL before insertion or after insertion. In addition, the cleats 300 would allow the surgeon a choice of lens types or powers to insert and the surgeon could potentially clip one or more lenses 200 onto the cleat 300. The cleats 300 would also allow for the replacement of a lens 200 as necessary due to a change in the power or type of lens 200 needed. A further aid to the surgeon would be to tint the cleats 300 and/or eyelets 400 such that they would be more visually identifiable to the surgeon during the operation.

With reference to FIG. 4, an alternative embodiment of the frame is shown which has a four point structure or a generally “stacked L” shape and contains four feet 121. This embodiment has two “V”-shaped structures, the arms of which are composed of two flexible support members 190. In this embodiment, the cleats 300 are attached to or an integral part of the flexible support member 190.

The embodiment shown in FIG. 5 is an alternative embodiment of the three-point frame structure which has two “V”-shaped structures. Both “V”-shaped structures have as one arm a lens mounting member 150 and as the other arm, a flexible support member 190. This figure shows the lens 200 attached by the eyelets 400 to the haptic cleats 300. Both haptic cleats 300 are attached to or are an integral part of, the lens mounting members 150.

FIG. 6 shows an alternate embodiment of the three-point frame structure which has one “V”-shaped structure with an angle αof about 90°, which is configured in a different direction from those of the embodiments in FIGS. 2 and 5.

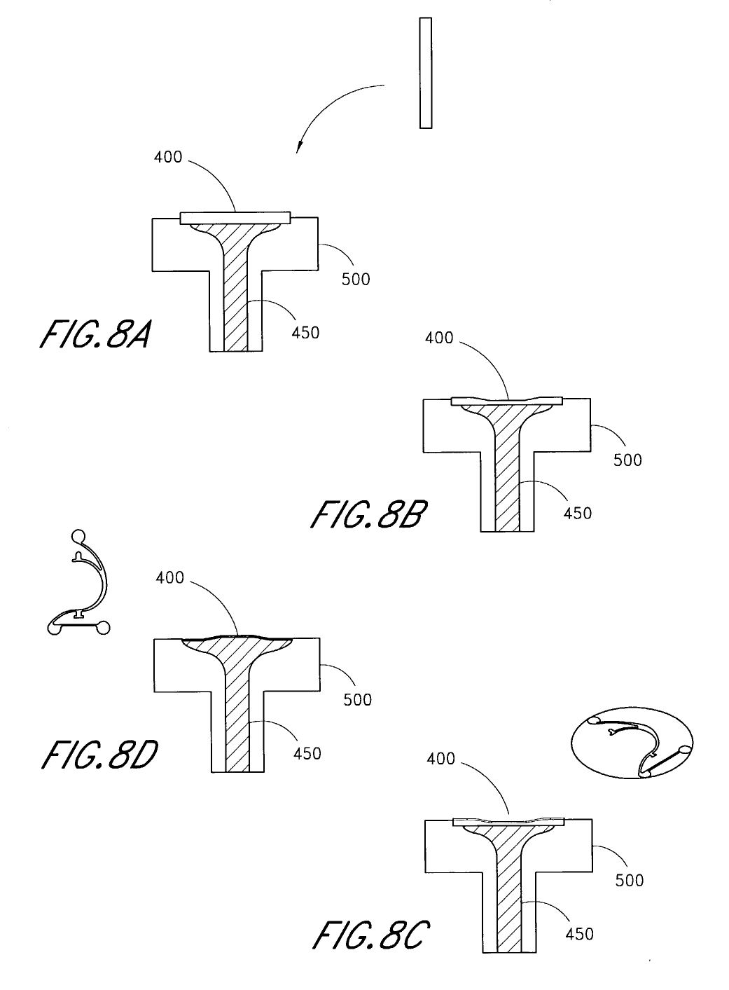

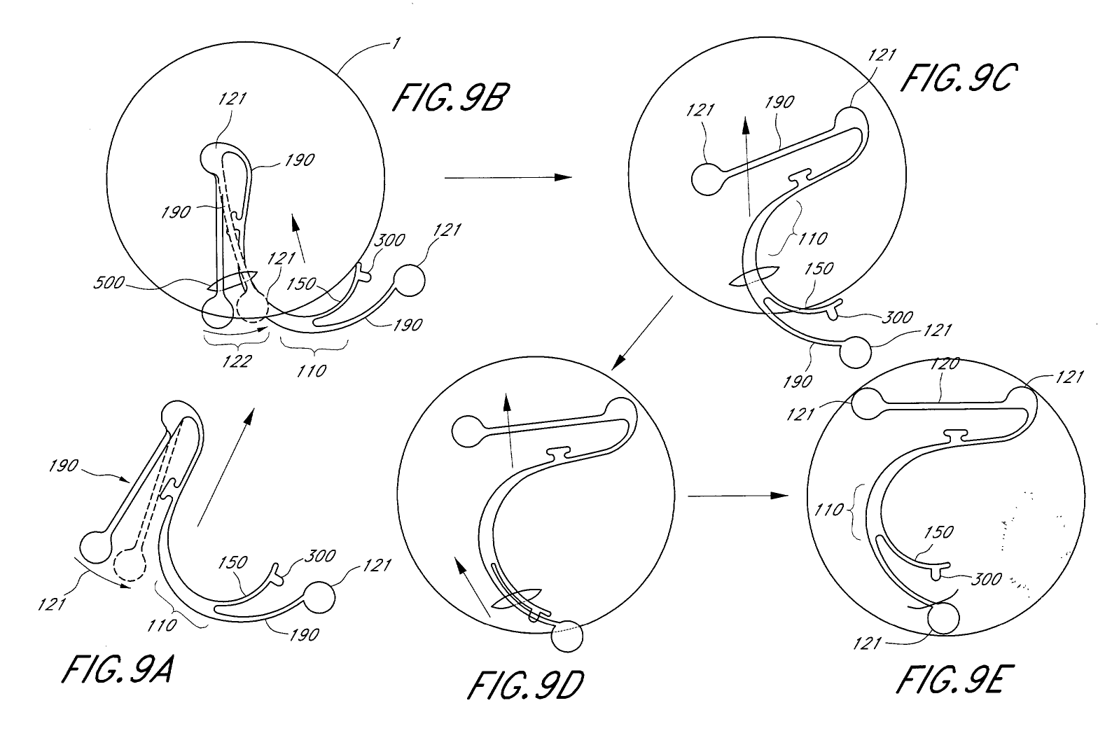

With reference to FIGS. 7 and 8A-8D, the method of producing the invention of the preferred embodiment will be described. Generally, the frame haptic is CNC milled and lathe cut from a 2 to 3 mm PMMA sheet down to about a 0.2 to about a 0.3 mm shaped thickness, preferably to about a 0.25 mm shaped thickness and from about 0.10 mm up to about 0.25 mm width segments.

The process of CNC milling and lathe cutting is now described: A round blank 400 from about 13 mm to 19 mm diameter is cored from a sheet of PMMA about 1 mm to 4 mm thick. The blank 400 is assembled onto a blocker 500 (see FIG. 8A). The blank 400 is held in place by injecting hot liquid wax 450 into the blocker and allowing it to cool and harden. The blocker 500 with the blank 400 attached is put into a CNC lathe and one side (the posterior side of the lens vault 180) is machined (concave) (see FIG. 8B). With continued reference to FIG. 8, in the next step, the blocker 500 is removed from the lathe and put in a CNC mill and the haptic shape 110 is machined on this same side, preferably about 0.5 mm in depth, but is not cut completely through the blank 400 (see FIG. 8C). The blank 400 is then removed from the blocker 500 and turned over so that the premachined haptic shape 110 is on the bottom (see FIG. 8D). The blank 400 is held into the blocker 500 again with wax 450. The haptic 110 is sculpted down to a final thickness of about 0.15 mm to 0.3 mm, thus separating it from the blank 400. At this point, the vaulting 180 is again produced in the haptic 110. FIG. 7 shows a side view of the machined frame haptic 110 which has an unstressed manufactured vault 180 of from about 0.25 to 1.3 mm, preferably, from about 0.50 to 1.0 mm. In the last step the haptic 110 is cleaned and inspected for flaws and the bevel 302 is machined into the backside of the cleats 300 (number 302 in FIG. 2).

The thin frame haptic 110 is typically next polished to remove any rough edges. The preferred method of polishing involves abrasive tumble agitation polishing with a media comprising glass beads and water with an abrasive.

In the preferred embodiment, the frame haptic 110 is polymethylmethacrylate which has a tensile modulus of about 450,000 psi (using test method D 638 of the ASTM). In the preferred embodiment the feet 121 are identical, but, non-identical feet 121 configurations can be paired for use in an alternative embodiment when necessary. The narrowness of the frame haptic segments 110 contributes to its springiness and lightness which is advantageous in that the IOL is less likely to be disrupted from its initial position, but still be able to automatically adjust to a non-round corneal/iris angle diameter without excessive forces. This allows for the fact that the cornea may be elliptical or oblong. The haptic 110 can be as narrow as about 0.05 mm to about 0.25 mm, preferably about 0.18 mm or between about 0.15 mm and about 0.22 mm.

The lenses of these designs are typically about half the weight of a standard lens and can be between 2 to 10 milligrams and as low as 1 milligram in weight in air and about 10% of this when in the aqueous of the eye. Preferably the lens is flexible but may be made of a hard, stiff, low memory material. However, in the preferred embodiment, the lens is made of silicone and the chosen silicone can be as low as 15 shore A. The index (N) value would be 1.430 to 1.460 or flexible acrylic N=1.45 to 1.47.

FIGS. 9A-E illustrate how the haptic frame can be manipulated through a very small incision by flexing of the arms of the “V”-shaped structures. In FIGS. 9A-E, the combination of the generally “L”-shaped haptic and bendable “V”-shaped structures allows for insertion through a very small incision 500 by flexing the arms of the “V” shaped structures of the haptic as it is manipulated and moved into the eye 1. In fact, in most embodiments, the arms of the “V” shaped structures are induced to bend by the living tissue of the incision as they are manipulated through the incision and are temporarily pushed together or over each other by the tissue of the eye. Alternatively a forceps or a push-rod configured probe can be used to aid in the bending of the “V”-shaped structures. It can be envisioned that the haptic can be manipulated into the eye 1 by holding onto the bottom of the “V”-shaped structure, between the arms, as it goes into the eye and the eye tissue itself will cause the arms of the “V” to flex up to or over themselves. FIG. 9A shows the haptic initially being inserted into the incision starting at the “corner” α. The first “V”-shaped structure can be manually flexed with a forceps or similar instrument, or optionally, as the foot 121 is manipulated into the incision, the eye tissue will automatically cause the two arms of the “V” shape to move together or over each other (see FIG. 9B). At this point (FIG. 9C) the haptic 110 is manipulated such that the “corner” is inserted and the haptic 110 is rotated until the short arm of the haptic 110 lines up with the edge of the eye 1 and the long arm is about perpendicular to the incision. The long arm is inserted by pushing the haptic 110 straight in (9C). In FIG. 9D, the arms of the second “V”-shaped portion are flexed or bent over or onto themselves and manipulated through the incision. Once again, it is envisioned that the tissue of the eye 1 at the incision 500 will automatically urge the arms of the “V”-shaped portion to bend. Because of the position of the incision in the eye 1, the last step (FIG. 9E) may require a slight axial shortening of the haptic 110 by slightly springing it inwardly to be fully inserted into the eye 1. Such springing is distinguished from the distortions, such as folding bending or rolling, normally used to introduce a lens into the eye. It can be envisioned that a number of different “L” or stacked “L” shapes could be used to produce such a haptic 110 as well as a number of different “V”-shaped structures. FIG. 4 shows an example of the stacked “L” shape. FIG. 6 shows an example of a different “V”-shaped structure in which one “V” is configured alternately. This type of haptic 110 would likely require a manual flexing of the initial “V” shape into the incision 500.

In FIGS. 9A-E, the haptic 110 is inserted into the very small opening 500 and positioned in the eye as desired. In FIG. 10, the optic 200 is rolled or folded as needed and inserted into the eye with forceps and attached to the distal cleat 300 from the opening, shown in FIG. 10A-C. The second eyelet 400 on the proximal side of the optic 200 is then attached to the cleat 300 closest to the opening (FIG. 10C) (see also Example 1). The movements which are used to attach the eyelet 400 of the optic 200 to the cleat 300 of the haptic 110 in this embodiment can be envisioned as 3 motions: an up, a down, and an in. This assembly process, even though as described herein as a series of cartesian (angular) motions, may be very smooth. The design provides for a skilled and practiced clinician to perform this motion in a deliberate, symphonic motion, only lasting a few seconds or moments. Additionally, extra eyelets 400 may be placed on the optic 200 so that it can be rotationally un-hooked, rotated or even turned over and re-attached in a new location using different eyelets 400.

In most previous IOL's, the lenses or optics 200 have predominantly been round. However, it can be envisioned that the lens 200 in the preferred embodiment of the present IOL 10 can be of many shapes. For example, the lens 200 may be oval, which would advantageously make the IOL narrower. Alternatively, the lens 200 may be segmented or chopped at one side to reduce the overall width of the IOL 10. The optic 200 may also have a parallelogram shape or even a trapezoid shape again allowing for a reduction in overall width. In this case the IOL 10 may have up to four eyelets 400 or even up to six or eight for rotational adjustment.

FIG. 11 shows the product of the preferred embodiment of the invention packaged in two separated parts 110 and 200 to be assembled into the eye.

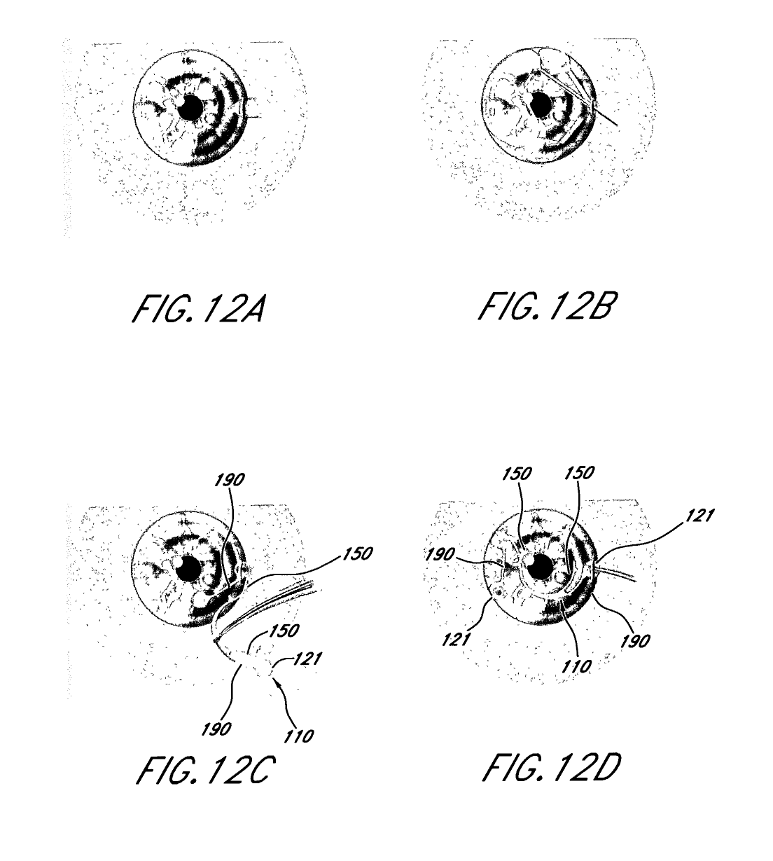

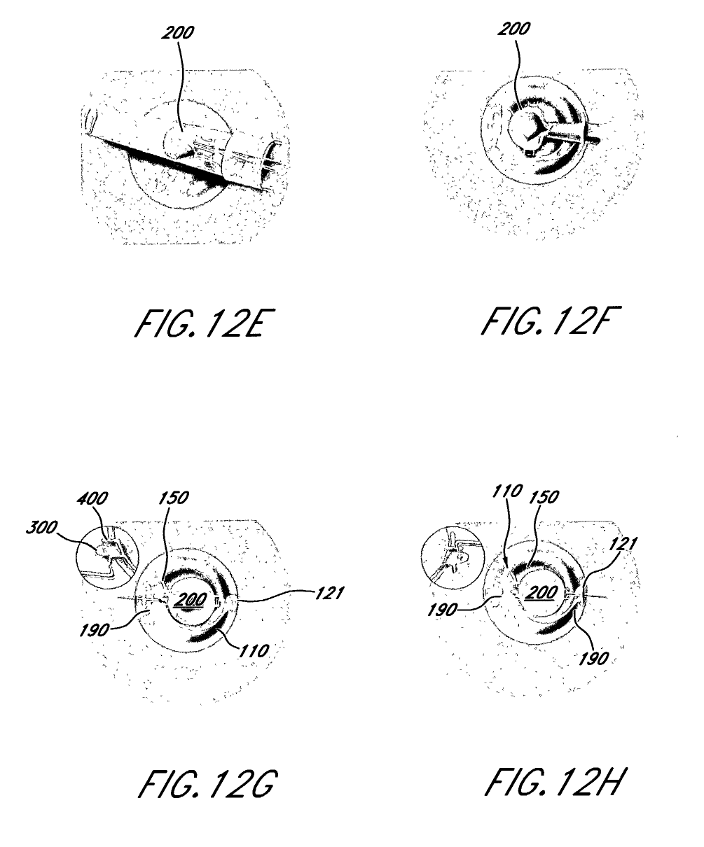

An embodiment of the surgical technique is shown in FIGS. 12A-H. The incision is performed as in FIG. 12A, the viscoelastic material is inserted in FIG. 12B, a haptic 110 of the preferred embodiment is snaked or manipulated into the incision. In this embodiment, the haptic 110 is inserted with little or no use of flexion of the lens mounting members 150 and/or the flexible support member 190. In FIG. 12D the last foot 121 is inserted. The optic 200 is then rolled and inserted in FIGS. 12E and 12F. The distal eyelet 400 is attached as in FIG. 12G and the proximal eyelet is attached as in FIG. 12H.

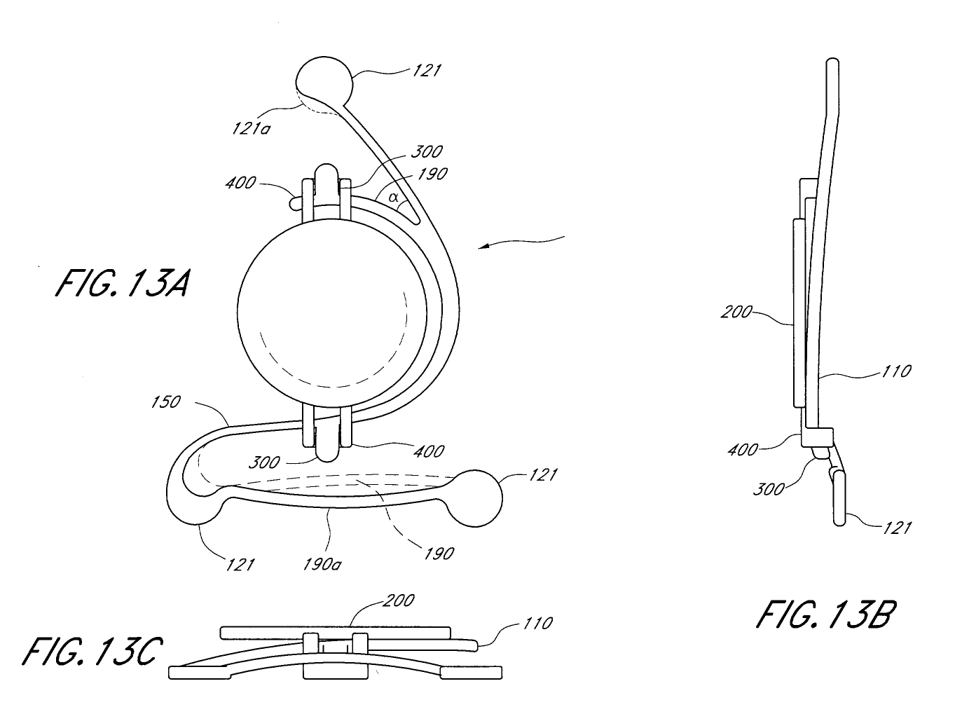

In order to increase the ease of attachment of the optic 200 to the haptic 110, a further embodiment is shown in FIG. 13A-C. This embodiment allows more space between the arms of the “V”-shaped structures by rounding them as in the flexible support member 190 to produce 190a. The embodiment of the haptic from FIG. 5 is shown in dashed lines for comparison. Alternatively or in addition, the foot 121 of the flexible support member 190 may be formed as an oval 121a to produce more space. This allows the surgeon to attach the eyelet 400 to the cleat 300 quickly and easily. In FIG. 13B the sideview of the IOL shows that the vaulted shape of the haptic 110 also provides a clearer area for attachment of the eyelet 400 to the cleat 300. The vaulted structure ensures that the feet 121 are at a different level than the lens 200. This can be seen further in FIG. 13C in which the IOL is shown from a front sideview.

An alternative embodiment of the IOL is shown in FIGS. 14-19. This embodiment is advantageously designed to fit into the posterior chamber 15 of the eye as shown in FIG. 14. However, the IOL may also be suitable for use in the anterior chamber 16 of the eye. In this embodiment, “V” shaped includes 2 members that come together at less than 90° even if there is a third member and in this regard including a curved “V” or a “U” or a “C”. With reference to FIG. 15A-C, the separable multi-part IOL arranged and configured in accordance with certain features, aspects and advantages of the present invention will be described in detail. FIG. 15 is a plan view of three embodiments of the thin frame haptic 110 of the plural part IOL 10. With reference to FIG. 15A, in this embodiment the thin frame haptic 110 includes at least two flexible support members 190. The thin frame/haptic 110 comprises at least two areas which come in contact with the eye tissue. The two feet 121 and multiple flexible support members 190 are arranged in an approximate forward or backward “S”-shape with at least one rounded “V”-shape. By “V” shape, it is envisioned that there is at least one “corner” or “angle” alpha (α) which is as great as 90° or less, but preferably from about 15 to 50°, more preferably between 30 and 45° (angular degrees), however, the corners may be rounded up to and including a “C” shape. In addition, there is at least one lens mounting member 150 which is structurally immobilized and produces, when paired with a flexible support member 190, a rounded “V”-shaped structure. It is envisioned that the flexible support member 190 can function as a lens mounting member 150, allowing the lens to be attached directly to the flexible support member 190. The combination of the flexible support member 190 with the lens mounting member 150, produces a rounded “V”-shaped structure, the arms of which can be flexed during insertion through an incision in the eye 1. The arms of the rounded “V” shaped structure can include one flexible support member 190 and one lens mounting member 150, two flexible support members 190 or mixtures of the two. The flexible, rounded “V” shaped structure allows the haptic 110 to be inserted into a very small incision by bending the haptic elements (or arms) and, more specifically, by bending the flexible support member 190 of the “V”-shaped structure, up to or over, the structurally immobilized lens mounting member 150. However, the lens may also be snaked or moved into the eye without flexing the support members 190. The maximum dimension of each section along the length of the haptic 110, when bent, is less than the incision. The haptic can be temporarily bent up to about 1 to about 1.5 mm or up to about 3 mm as the frame is passed through the incision.

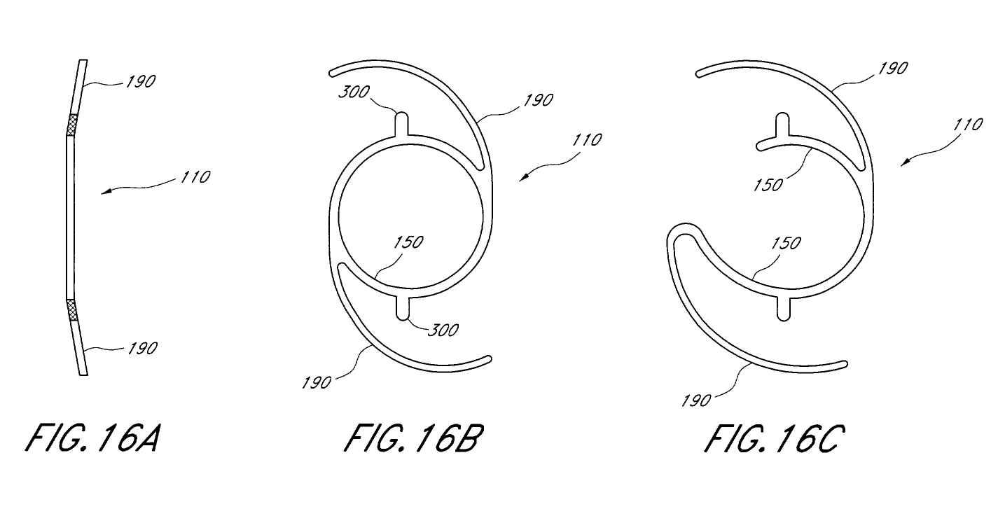

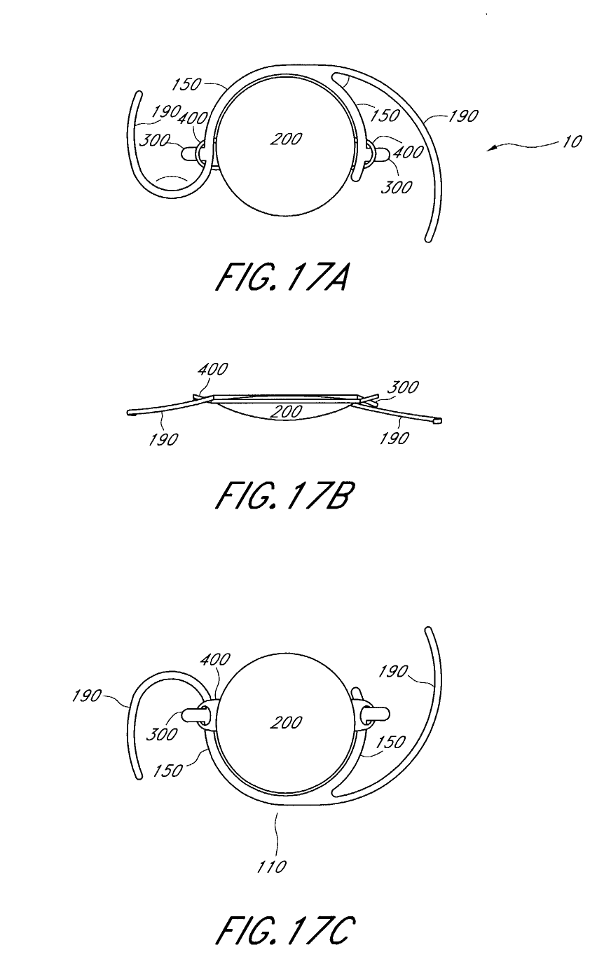

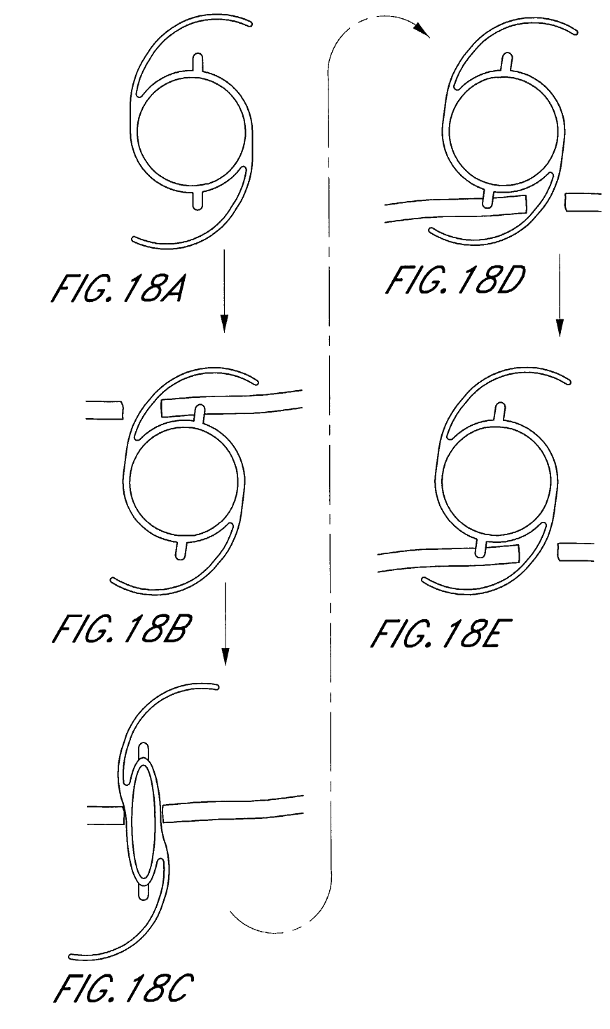

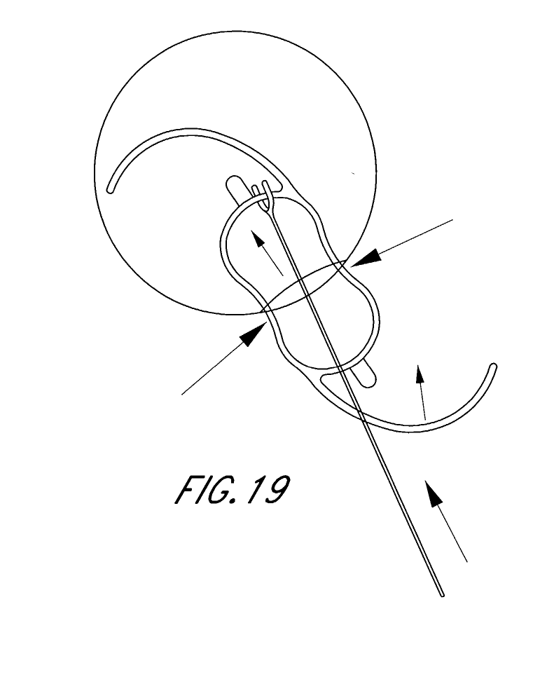

Alternative embodiments, shown in FIGS. 15B and C may have “V” shaped structures which are more or less rounded with respect to the angle α. With reference to FIGS. 16A-C, a further embodiment includes a closed configuration, FIG. 16B, in which the lens mounting member 150 is completely closed. Alternatively, the lens mounting member 150 may be partially closed. FIG. 16C is an alternative open embodiment in which the lens mounting member 150 is open. FIG. 16A is a sideview of either embodiment, showing the ramping of the support members 190 which allows for ease of attachment of the lens 200 to the cleats 300. FIGS. 17A-C show the open embodiment from FIG. 16C with the lens 200 attached. FIGS. 17A and C show that the lens 200 may be attached to a haptic 110 above or below the cleat to place the optic within the vault or outside the vault. The sideview, FIG. 17B, corresponds to the arrangement shown in FIG. 17A. FIGS. 18A-E show the insertion of the closed embodiment (shown in FIG. 16B) into the eye 1. The closed haptic, FIG. 18A is inserted by first snaking or manipulating the support member 190 (the leading haptic) into the incision up to the closed lens mounting member 150 (FIG. 18B). The lens mounting member 150 is flexible and can be flexed up to or over the opposite side of the lens mounting member 150 (see FIG. 18C). The opposite support member 190 is then snaked through in FIG. 18D (the trailing haptic) and the foot 121 is flexed into the eye. FIG. 19 is a close-up of the process of moving the closed lens mounting members 150 through the incision. This Figure shows that a tool may be used to push the leading haptic through the incision. It can be seen that the lens mounting member 150 is flexed radially inwardly as it passes through the incision. The eye tissue will automatically cause the two sides of the lens mounting members 150 to move together or over each other.

In FIGS. 20A and B, the open embodiment of the haptic 110 shown in FIG. 16C is shown being manipulated into the eye 1. The leading haptic of this embodiment is typically snaked into the incision. The trailing haptic is pushed through the incision and the lens mounting member 150 is flexed over the trailing support member by the eye tissue. In this figure a tool is used to push the trailing lens mounting member 150 and support member 190 into the eye 1. In FIG. 20B, the tool is then used to push the trailing support member 190 completely through the incision.

The last step of assembling the optic 200 onto the haptic 110 may be accomplished as it was in FIGS. 10A-C with an alternate embodiment. As with the alternate embodiment, it is to be understood that the optic 200 may be assembled onto the haptic 110 as it is being inserted into the eye 1. Alternatively, the optic 200 may be partially or completely assembled onto the haptic before it is inserted into the eye.

### EXAMPLES

Preferred embodiments of the intraocular lens and the insertion of the intraocular lens will now be described in the Examples.

An embodiment of the haptic and optic was produced using PMMA haptics of a variety of sizes to fit any eye. Because the anterior chamber can be hard to fit correctly, due to its uneven nature, the haptic may require replacement with a slightly different sized haptic to correctly fit. Thus, 12.0, 12.5, 13, and 13.5 mm haptics were initially available. At each size, the haptics conform to the anterior chamber without compressing the 5.5 mm silicone optic. Initially, a haptic modeled on the embodiment shown in FIG. 5 was constructed and used in the clinical trials.

### Example 1

### Insertion of the Two Part IOL Into the Eye

A 1.5 mm incision is made near the limbus of the eye. Viscoelastics are then injected into the anterior chamber. The frame is inserted as shown in FIGS. 9A-E. Then the lens is inserted and attached as shown in FIGS. 10A-C: the surgeon grasps the folded optic with the outside (distal) eyelet leading forward (see FIG. 10A). The surgeon then pushes the lens through the incision, lets the lens unfold and manipulates it to hook the eyelet onto the distal cleat of the frame (see FIG 10B). Then, the surgeon slowly opens the forceps while maintaining slight tension. The lens is then grasped near or onto the closest eyelet (proximal) and stretched and pulled over the proximal cleat of the frame (see FIG 10C).

The intraocular lens was implanted into 6 patients in Spain in a Clinical Trial. All patients experienced an increase in best-corrected visual acuity of 1-2 Snellen lines. The results of 3 patients will be presented in more detail in Example 2.

### Example 2

### Clinical Trials of the Intraocular Lens

An lens according to the present invention with the trademark Kelman Duet Implant™ was used. The eyes of three patients were implanted with the Kelman Duet Implant™ by Dr. Jorge L. Alio at the Instituto Oftalmologico De Alicante in Spain. The patients were followed for 3 to six months. A 10.5D optic and a 12.5 mm frame were used as follows:

The surgery was performed as in FIG. 12A-H (see also FIGS. 9 and 10) and was performed either to compensate existing astigmatism (patient 3) or not to increase existing astigmatism (other patients). A 1 mm incision was made at 3 o'clock and a 3 mm incision at 9 o'clock as in FIG. 12A. Viscoelastic was introduced (being careful not to allow it to go posterior to the iris) (see FIG. 12B). In FIG. 12C, the haptic was snaked into the anterior chamber. The correct angle placement was verified with gonioscopy as in FIG. 12D. The optic was loaded right side up, with tabs folded upwards as in FIG. 12E. FIG. 12F shows that the optic emerged with the correct orientation. In FIG. 12G the first tab engaged the projection and in FIG. 12H, the second tab engaged the projection. When the surgery did not include Lasik, it was found that the assembly of the lens onto the frame of the invention could be done in 15 seconds to 4 minutes total.

Lasik was performed to solve the remaining astigmatism and residual myopia (as only 10.5 D IOLs were available) and to achieve good visual acuity with no correction. Patient 1 even with a −2 cylinder did not require Lasik or addition correction to achieve good vision. Glare was reported only when it was asked for the purpose of the questionnaire, otherwise the patients did not complain about glare.

Although the performance of Lasik secondary to a Phakic IOL implantation could lead to a risk of loss of visual acuity, it was not observed in the three cases. The lens position was checked at three months and remained in proper position; the lens was very stable due to its independent optic. The endothelial counts remained stable, thus, the IOL did not activate cellular multiplication.

Because patient 1 was implanted with a frame that was too large for that patient's eye, replacement of the frame was performed by leaving the optic inside of the anterior chamber and removing the haptic in a reverse of FIGS. 14C and D. This proves the interchangeability of the lens and haptic, allowing the surgeon to remove and replace either or both with a minimum of trauma to the eye and patient. The results for each patient are shown in Tables 1-3.

In the tables: ACD=anterior chamber depth, IOP=intraocular pressure, UCVA =uncorrected visual acuity, BCVA=best corrected visual acuity, W-to-W=white to white. M1=the first month, M3=the third month, M6=the sixth month.

TABLE 1
Results of the implantation of the IOL in Patient 1

| DOB Apr. 4, 1978 | PreOp | D1/W1 | M1 | M3 | M6 |
|---|---|---|---|---|---|
| W-to-W | 12 | | | | |
| Endothelial | 2752 | | 2957 | — | 2256 |
| Cells | | | | | |
| Keratometry | K1 | | | | |
| | 45.25 | | | | |
| | K2 | | | | |
| | 46.25 | | | | |
| ACD | 2.64 | | | | |
| IOP | 12 | | 34 (Hypertension | 14 | 20 |
| | | | due to | | |
| | | | corticosteroids | | |
| UCVA | 20/30 | 20/30 | 20/30 | 20/30 | 20/20 |
| BCVA | 20/20 | 20/30 | 20/30 | 20/20 | 20/20 |
| Refraction | −9.5/ | −/−4 | −/−.05 | −/−2 | −/−2 |
| | −1.75 | | | | |
| Glare | | +++ | ++++ | ++++ | 0 |
| Pupil | | Deform. | Deform. (Haptics | Deform. (Iris | OK |
| | | | touch iris) | pigments) | |
| Patient | | 4/10 | 4/10 | 4/10 | 7/10 (No |
| Satisfaction | | | | | correction) |

↑
Exchange of
Frame (12 mm)

TABLE 2
Results of the implantation of the IOL in Patient 2

| DOB | | | | | |
|---|---|---|---|---|---|
| Mar. 19, 1968 | PreOp | D1 | W1 | M1 | M3 |
| W-to-W | 12.5 | | | | |
| | (Mean) | | | | |
| Endothelial | 2526 | | | 1945 | 2194 |
| Cells | | | | | |
| Keratometry | K1 41. | | | | |
| | K2 43.25 | | | | |
| ACD | 3.91 | | | | |
| IOP | 12 | 12 | 18 | 18 | 18 |
| UCVA | 20/70 | 20/100 | 20/200 (double | 20/100 | 20/40 |
| | | | vision) | | |
| BCVA | 20/20 | 20/70 | 20/20 | 20/20 | 20/40 |
| Refraction | −12/−1.75 | +1/−0.5 | −2.5/−1.5 | −2.5/−1.5 | — |
| | | (Early post op, | | | |
| | | corneal edema) | | | |
| Glare | | | +++ | +++ | +++ |
| | | | | | Subjective |
| Pupil | | Ovalization | OK | OK | OK |
| Patient | | 3/10 | 3/10 | 5/10 | 7/10 |
| Satisfaction | | | | | |

↑
LASIK

TABLE 3
Results of the implantation of the IOL in Patient 3

| DOB Jan. 1, 1970 | PreOp | D1/W1 | M1 | M3 | M6 |
|---|---|---|---|---|---|
| W-to-W | 11.5/12/5 | | | | |
| Endothelial | 2312 | | 2957 | — | 2773 |
| Cells | | | | | |
| Keratometry | K1 43 | | | | |
| | K2 45.25 | | | | |
| ACD | 3.33 | | | | |
| IOP | 12 | 14 | 21 | 12 | 13 |
| UCVA | <20/200 | 20/200 | 20/200 | 20/40 | 20/40 |
| BCVA | 20/50 | 20/40 | 20/40 | 20/50 | 20/40 |
| | | | | Tear film | |
| Refraction | −13.60/−4 | −2.25/−2.5 | −3.5/−2 | −0.5/−0.75 | −0.75/−0.75 |
| Glare | | 0 | 0 | 0 | 0 |
| Pupil | | Cycloplegia due | OK | OK | OK |
| | | to mydriatics? | | | |
| Patient | | 5/10 | | 7/10 | 7/10 |
| Satisfaction | | | | | |

↑
LASIK

Therefore, the description and examples show that the IOL of the present invention presents a number of advantages. It is inserted in two separate pieces significantly reducing the bulk so that the incision can be as narrow as 1 mm. The narrow shape of the haptic arms allows for very low forces when flexed reducing perceptible sensitivity or irritating trauma which reduces corneal chafing and pupilary block. The disc shaped support feet on the frame are similar to older designed plate lenses and will minimize synechiae. Lastly, it can be used in a phakic or aphakic eye.

One advantage of the present invention is that because the lens is a multi-part assembly, the ideal properties of each part of the IOL can be retained. For example, the haptic is ideally more rigidly springy with high memory and can be constructed to fit into a very narrow incision. In addition, Since the haptic is not rigidly connected to the optic and the connections themselves allow for some movement, the entire length of the haptic is available for flexure. This can be envisioned as being comparable to the flexibility in a long, flat, thin piece of steel. It has some flexibility if sufficiently thin. However, if the same piece of steel is only 2 inches long, it is considerably less flexible. The lens, although it is between 3 mm and 7 mm, including about 4 mm, about 5 mm and about 6 mm, can be inserted into a narrow incision because it is constructed of a more pliable and soft material and can be folded, squeezed or rolled, more than it could be with the attached haptic, to be inserted into a considerably smaller incision using forceps or an injector. Because of this, and because the overall mass of the separate parts is less than the total mass, a multi-part IOL allows for insertion into a much narrower incision, than an assembled lens.

The lens can be implanted into the eye using a variety of surgical implant techniques known in the art. Although the preferred embodiment is that the lens be implanted into the anterior chamber, it can be envisioned that the lens could also be implanted in the posterior chamber or the lens could be comprised of two or more optics.

Additionally, any combination of the materials used will result in a lens that can be sterilized by a variety of standard methods such as ethylene oxide (ETO) or steam autoclaving at 250° F. or any other acceptable method.

Although the haptic of the preferred embodiment is described as being machine formed, it can also be envisioned that the haptic is molded or laser cut.

While this invention has been described with respect to various specific examples and embodiments, it is to be understood that the invention is not limited thereto and that it can be variously practiced within the scope of the following claims:

What is claimed is:

1. A multi-part intraocular lens (IOL) comprising:
an optic;
a haptic comprising:
at least one “V”-shaped element having a pair of legs and a square or rounded corner; and
at least two contact points for the eye each located on one of said legs or one of said corners; and
an attachment for the optic onto the haptic.

2. The multi-part intraocular lens of claim 1, comprising two “V”-shaped elements.

3. The multi-part intraocular lens of claim 1, wherein said “V”-shaped element is straight.

4. The multi-part intraocular lens of claim 1, wherein said “V”-shaped element is rounded.

5. The multi-part intraocular lens of claim 1, wherein said attachment comprises a cleat and an eyelet wherein said eyelet comprises an eyelet aperture.

6. The multi-part intraocular lens of claim 5, wherein said cleat is a part of said haptic.

7. The multi-part intraocular lens of claim 5, wherein said eyelet is a part of said lens.

8. The multi-part intraocular lens of claim 5, wherein said cleat is chamfered.

9. The multi-part intraocular lens of claim 5, wherein said eyelet is offset or angled to hook under said cleat.

10. The multi-part intraocular lens of claim 5, wherein said eyelet is a filament.

11. The multi-part intraocular lens of claim 5, wherein said eyelet is fabricated separately and attached to the lens.

12. The multi-part intraocular lens of claim 5, wherein said eyelet is tinted.

13. The multi-part intraocular lens of claim 5, wherein said cleat is fabricated separately and attached to the haptic.

14. The multi-part intraocular lens of claim 5, wherein said cleat is tinted.

15. The multi-part intraocular lens of claim 5, wherein said eyelet aperture has a diameter larger than the diameter of the cleat to allow for normal eye movements.

16. The multi-part intraocular lens of claim 6, wherein said haptic comprises at least two cleats.

17. The multipart intraocular lens of claim 8, wherein said lens comprises at least two eyelets.

18. The multipart intraocular lens of claim 17, wherein said lens comprises multiple eyelets to allow for rotation of the lens.

19. The multi-part intraocular lens of claim 1, wherein there are two or more attachments.

20. The multi-part intraocular lens of claim 1, wherein the two or more attachments are asymmetrical.

21. The multi-part intraocular lens of claim 1, wherein at least one of said legs of at least one of said “V”-shaped elements is sufficiently flexible to move the other one of said legs of said at least one of said “V”-shaped elements..

22. The intraocular lens of claim 1, wherein said haptic is composed of a material selected from the group consisting of: polyimide, polyetheretherketone, polycarbonate, polymethylpentene, polyphenylsulfone, polymethylmethacrylate (PMMA), polypropylene, polyvinylidene fluoride, polysulfone, and polyethersulfone.

23. The intraocular lens of claim 22, wherein said polyimide is KAPTON.

24. The intraocular lens of claim 22, wherein said haptic is composed of polymethylmethacrylate (PMMA).

25. The intraocular lens of claim 22, wherein said haptic has a modulus of elasticity of about 450,000 psi/inch.

26. The intraocular lens of claim 1, wherein said haptic has a modulus of elasticity of 100,000 to 500,000 psi.

27. The intraocular lens of claim 1, wherein said haptic is less than about 0.01 inches thick.

28. The intraocular lens of claim 1, wherein said haptic is machine-formed.

29. The intraocular lens of claim 1, wherein said haptic is laser cut.

30. The intraocular lens of claim 1, wherein said haptic is molded.

31. The intraocular lens of claim 1, wherein said haptic has a hardness of about 90 to 95 shore M.

32. The intraocular lens of claim 1, wherein said haptic is sized for a particular eye, and wherein one of said legs of said haptic is larger than the space within said particular eye.

33. The intraocular lens of claim 32, wherein the diameter of said haptic is up to about 1 mm greater than that of said particular eye.

34. The intraocular lens of claim 32, wherein the diameter of said haptic is between about 0.3 and 0.6 mm greater than that of said particular eye.

35. The intraocular lens of claim 32, wherein the diameter of said haptic is between about 0.4 and 0.5 mm greater than that of said particular eye.

36. The intraocular lens of claim 1, wherein said optic is selected from the group consisting of a refractive lens, a monofocal lens, a toric lens, an aspheric lens, a bifocal lens, an interference lens, a positive lens, a negative lens, a standard power monofocal lens, a multi-focal spheric lens, a multiple optic lens, an interference lens, a thin lens, a radially non-symmetrical lens, a laterally non-symmetrical lens and a defocusing lens.

37. The intraocular lens of claim 1, wherein said optic may be inserted into the anterior or posterior chamber of the eye.

38. The intraocular lens of claim 1, wherein the entire length of the haptic is available for flexure.

39. A multi-part intraocular lens, comprising:
a haptic with at least two “V” shaped elements;
a separate optic; and
an attachment for said optic which permits said optic to be attached to said haptic within the eye.

40. The multi-part intraocular lens of claim 39, wherein at least one of said “V”-shaped elements is rounded.

41. The multi-part intraocular lens of claim 39, wherein at least one of said “V”-shaped elements is straight.

42. The multi-part intraocular lens of claim 39, wherein said attachment comprises a cleat and an eyelet wherein said eyelet comprises an eyelet aperture.

43. The multi-part intraocular lens of claim 42, wherein said cleat is a part of said haptic.

44. The multi-part intraocular lens of claim 42, wherein said eyelet is a part of said lens.

45. The multi-part intraocular lens of claim 42, wherein said cleat is chamfered.

46. The multi-part intraocular lens of claim 42, wherein said eyelet is offset or angled to hook under said cleat.

47. The multi-part intraocular lens of claim 42, wherein said eyelet is a filament.

48. The multi-part intraocular lens of claim 42, wherein said eyelet is fabricated separately and attached to the lens.

49. The multi-part intraocular lens of claim 42, wherein said eyelet is tinted.

50. The multi-part intraocular lens of claim 42, wherein said cleat is fabricated separately and attached to the haptic.

51. The multi-part intraocular lens of claim 42, wherein said cleat is tinted.

52. The multi-part intraocular lens of claim 42, wherein said eyelet aperture has a diameter larger than the diameter of the cleat to allow for normal eye movements.

53. The multi-part intraocular lens of claim 43, wherein said haptic comprises at least two cleats.

54. The multipart intraocular lens of claim 45, wherein said lens comprises at least two eyelets.

55. The multipart intraocular lens of claim 54, wherein said lens comprises multiple eyelets to allow for rotation of the lens.

56. The multi-part intraocular lens of claim 42, wherein there are two or more attachments.

57. The multi-part intraocular lens of claim 42, wherein the two or more attachments are asymmetrical.

58. The multi-part intraocular lens of claim 42, wherein at least one of said legs of at least one of said “V”-shaped elements is sufficiently flexible to move the other one of said legs of said at least one of said “V”-shaped elements..

59. The intraocular lens of claim 42, wherein said haptic is composed of a material selected from the group consisting of: polyimide, polyetheretherketone, polycarbonate, polymethylpentene, polyphenylsulfone, polymethylmethacrylate (PMMA), polypropylene, polyvinylidene fluoride, polysulfone, and polyethersulfone.

60. The intraocular lens of claim 59, wherein said polyimide is KAPTON.

61. The intraocular lens of claim 59, wherein said haptic is composed of polymethylmethacrylate (PMMA).

62. The intraocular lens of claim 59, wherein said haptic has a modulus of elasticity of about 450,000 psi/inch.

63. The intraocular lens of claim 42, wherein said haptic has a modulus of elasticity of 100,000 to 500,000 psi.

64. The intraocular lens of claim 42, wherein said haptic is less than about 0.01 inches thick.

65. The intraocular lens of claim 42, wherein said haptic is machine-formed.

66. The intraocular lens of claim 42, wherein said haptic is laser cut.

67. The intraocular lens of claim 42, wherein said haptic is molded.

68. The intraocular lens of claim 42, wherein said haptic has a hardness of about 90 to 95 shore M.

69. The intraocular lens of claim 42, wherein said haptic is sized for a particular eye, and wherein one of said legs of said haptic is larger than the space within said particular eye.

70. The intraocular lens of claim 69, wherein the diameter of said haptic is up to about 1 mm greater than that of said particular eye.

71. The intraocular lens of claim 69, wherein the diameter of said haptic is between about 0.3 and 0.6 mm greater than that of said particular eye.

72. The intraocular lens of claim 69, wherein the diameter of said haptic is between about 0.4 and 0.5 mm greater than that of said particular eye.

73. The intraocular lens of claim 42, wherein said optic is selected from the group consisting of a refractive lens, a monofocal lens, a toric lens, an aspheric lens, a bifocal lens, an interference lens, a positive lens, a negative lens, a standard power monofocal lens, a multi-focal spheric lens, a multiple optic lens, an interference lens, a thin lens, a radially non-symmetrical lens, a laterally non-symmetrical lens and a defocusing lens.

74. The intraocular lens of claim 42, wherein said optic may be inserted into the anterior or posterior chamber of the eye.

75. A method for introducing an intraocular lens into a very small incision in an eye, comprising:
inserting the haptic of claim 1 into the eye;
inserting the optic of claim 1 into the eye separate from said haptic; and
attaching said optic onto said haptic within the eye using the attachment of claim 1.

76. The method of claim 75 wherein said insertion of said haptic into the eye is by flexing or bending said legs of said “V”-shaped elements toward each other.

77. The method of claim 75, wherein said haptic is inserted first.

78. The method of claim 75, wherein said optic is inserted first.

79. The method of claim 75, further comprising removing said optic and replacing it with a different optic.

80. The method of claim 75, further comprising removing said optic and repositioning it within the eye.

81. The method of claim 80, wherein said repositioning comprises rotational repositioning for correction of astigmatism.

82. The method of claim 81, wherein said repositioning comprises turning the optic over.

83. The method of claim 75, further comprising adding a second optic.

84. The method of claim 75, further comprising removing said haptic and replacing it with a different haptic.

85. The method of claim 75, further comprising removing said haptic and repositioning it within the eye.

86. The method of claim 75, wherein said optic is formed of a relatively lower modulus material than said haptic.

87. The method of claim 75, wherein said optic is attached to said haptic with a stretchable attachment.

88. The method of claim 75, further comprising partially assembling said optic onto said haptic during insertion.

89. The method of claim 74, further comprising assembling said optic onto said haptic prior to insertion.

90. A method for introducing an intraocular lens into a very small incision in an eye, comprising:
inserting the haptic of claim 39 into the eye;
inserting the optic of claim 39 into the eye separate from said haptic; and
attaching said optic onto said haptic within the eye using the attachment of claim 39.

91. The method of claim 90 wherein said insertion of said haptic into the eye is by flexing or bending said legs of said “V”-shaped elements toward each other.

92. The method of claim 90, wherein said haptic is inserted first.

93. The method of claim 90, wherein said optic is inserted first.

94. The method of claim 90, further comprising removing said optic and replacing it with a different optic.

95. The method of claim 90, further comprising removing said optic and repositioning it within the eye.

96. The method of claim 95, wherein said repositioning comprises rotational repositioning for correction of astigmatism.

97. The method of claim 95, wherein said repositioning comprises turning the optic over.

98. The method of claim 90, further comprising adding a second optic.

99. The method of claim 90, further comprising removing said haptic and replacing it with a different haptic.

100. The method of claim 90, further comprising removing said haptic and repositioning it within the eye.

101. The method of claim 90, wherein said optic is formed of a relatively lower modulus material than said haptic.

102. The method of claim 90, wherein said optic is attached to said haptic with a stretchable attachment.

103. The method of claim 90, further comprising partially assembling said optic onto said haptic during insertion.

104. The method of claim 90, further comprising assembling said optic onto said haptic prior to insertion.

105. A haptic for an intraocular lens, comprising:
a first “V” shaped portion, having a maximum interior angle less than 90° and two arms; and
a second “V” shaped portion, having a maximum interior angle less than 90° and two arms, one of said arms joined to said first “V” shaped portion at said interior angle of said first “V” shape.

106. The haptic of claim 105 wherein at least one of said “V”-shaped portions are rounded.

107. The haptic of claim 105, wherein at least one of said “V”-shaped portions are straight.

108. The haptic of claim 105, further comprising at least one cleat for attachment to an optic.

109. The haptic of claim 108 wherein said cleat is chamfered.

110. The haptic of claim 108 wherein said cleat is fabricated separately and attached to the haptic.

111. The haptic of claim 108, wherein said cleat is tinted.

112. The haptic of claim 108, wherein said haptic comprises two or more cleats.

113. The haptic of claim 109, wherein said arms of said at least one of said “V”-shaped elements is sufficiently flexible to move the other one of said arms of said at least one “V” shaped elements.

114. The haptic of claim 109, wherein said haptic is composed of a material selected from the group consisting of: polyimide, polyetheretherketone, polycarbonate, polymethylpentene, polyphenylsulfone, polymethylmethacrylate (PMMA), polypropylene, polyvinylidene fluoride, polysulfone, and polyethersulfone.

115. The haptic of claim 114, wherein said polyimide is KAPTON.

116. The haptic of claim 114, wherein said haptic is composed of polymethylmethacrylate (PMMA).

117. The haptic of claim 109, wherein said haptic has a modulus of elasticity of about 450,000 psi/inch.

118. The haptic of claim 109, wherein said haptic has a modulus of elasticity of 100,000 to 500,000 psi.

119. The haptic of claim 109, wherein said haptic is less than about 0.01 inches thick.

120. The haptic of claim 109, wherein said haptic is machine-formed.

121. The haptic of claim 109, wherein said haptic is laser cut.

122. The haptic of claim 109, wherein said haptic is molded.

123. The haptic of claim 109, wherein said haptic has a hardness of about 90 to 95 shore M.

124. The haptic of claim 109, wherein said haptic is sized for a particular eye, and wherein one of said legs of said haptic is larger than the space within said particular eye.

125. The haptic of claim 124, wherein the diameter of said haptic is up to about 1 mm greater than that of said particular eye.

126. The haptic of claim 124, wherein the diameter of said haptic is between about 0.3 and 0.6 mm greater than that of said particular eye.

127. The haptic of claim 124, wherein the diameter of said haptic is between about 0.4 and 0.5 mm greater than that of said particular eye.

128. The haptic of claim 109, further comprising an eyelet.

129. A method of inserting a haptic into a patient's eye, comprising:
threading the haptic of claim 109 through an incision smaller than 2.0 mm into said eye, by bending the arms of two “V” shaped structures onto each other as they pass through said incision.

130. A method of inserting a haptic into a patient's eye, comprising:
threading said haptic through an incision smaller than 2.0 mm into said eye, by bending the arms of two “V” shaped structures onto each other as they pass through said incision.

131. The method of claim 130, wherein at least one of said “V”-shaped structures is rounded.

132. The method of claim 130, wherein at least one of said “V”-shaped structures is straight.

133. The method of claim 130, wherein said method takes less than about 10 minutes.

134. The method of claim 130, wherein said method takes less than about 5 minutes.

135. The method of claim 130, wherein said method takes less than about 2 minutes.

136. A method for introducing an intraocular lens haptic having first and second “V”-shaped elements into a very small incision in an eye, comprising:
flexing each arm of said first “V”-shaped element of said haptic next to or over each other and inserting said first “V”-shaped element into the eye; and
flexing each arm of said second “V”-shaped element of said haptic next to or over each other and inserting said second “V”-shaped element into the eye.

137. The method of claim 136, wherein said first “V”-shaped elements is rounded.

138. The method of claim 136, wherein said second “V”-shaped element is rounded.

139. The method of claim 136, wherein said first “V”-shaped element is straight.

140. The method of claims 136, wherein said second “V”-shaped element is straight.

141. The method of claim 136 wherein said “V”-shaped elements include plate-type haptics.

142. A method for introducing an intraocular lens haptic having first and second “V”-shaped elements into a very small incision in an eye, comprising:
flexing each arm of said first “V”-shaped element of said haptic next to or over each other and inserting said first “V”-shaped element into the eye; and
manipulating without bending said second “V”-shaped element into the eye.

143. The method of claim 142, wherein said first “V”-shaped elements is rounded.

144. The method of claim 142, wherein said second “V”-shaped element is rounded.

145. The method of claim 142, wherein said first “V”-shaped element is straight.

146. The method of claims 142, wherein said second “V”-shaped element is straight.

147. The method of claim 142 wherein said “V”-shaped elements include plate-type haptics.

148. A haptic for supporting an intraocular lens in an eye, and for insertion into said eye through a small incision, comprising:
a pair of arms, connected at one end to form a “V”-shape, at least one of said arms sufficiently flexible to permit said small incision to squeeze said pair of arms toward one another as said haptic is inserted through said small incision.

149. The haptic of claim 148, wherein said “V” shape is straight.

150. The haptic of claim 148, wherein said “V” shape is rounded.

151. The haptic of claim 148, further comprising at least one cleat for attachment to an optic.

152. The haptic of claim 148 wherein said cleat is chamfered.

153. The haptic of claim 148 wherein said cleat is fabricated separately and attached to the haptic.

154. The haptic of claim 148, wherein said cleat is tinted.

155. The haptic of claim 148, wherein said haptic comprises two or more cleats.

156. The haptic of claim 148, wherein said arms of said at least one of said “V”-shaped elements is sufficiently flexible to move the other one of said arms of said at least one “V” shaped elements.

157. The haptic of claim 148, wherein said haptic is composed of a material selected from the group consisting of: polyimide, polyetheretherketone, polycarbonate, polymethylpentene, polyphenylsulfone, polymethylmethacrylate (PMMA), polypropylene, polyvinylidene fluoride, polysulfone, and polyethersulfone.

158. The haptic of claim 157, wherein said polyimide is KAPTON.

159. The haptic of claim 157, wherein said haptic is composed of polymethylmethacrylate (PMMA).

160. The haptic of claim 150, wherein said haptic has a modulus of elasticity of about 450,000 psi/inch.

161. The haptic of claim 149, wherein said haptic has a modulus of elasticity of 100,000 to 500,000 psi.

162. The haptic of claim 149, wherein said haptic is less than about 0.01 inches thick.

163. The haptic of claim 149, wherein said haptic is machine-formed.

164. The haptic of claim 149, wherein said haptic is laser cut.

165. The haptic of claim 149, wherein said haptic is molded.

166. The haptic of claim 149, wherein said haptic has a hardness of about 90 to 95 shore M.

167. The haptic of claim 149, wherein said haptic is sized for a particular eye, and wherein one of said legs of said haptic is larger than the space within said particular eye.

168. The haptic of claim 167, wherein the diameter of said haptic is up to about 1 mm greater than that of said particular eye.

169. The haptic of claim 167, wherein the diameter of said haptic is between about 0.3 and 0.6 mm greater than that of said particular eye.

170. The haptic of claim 167, wherein the diameter of said haptic is between about 0.4 and 0.5 mm greater than that of said particular eye.

171. The haptic of claim 149, further comprising an eyelet.

172. A haptic for supporting an intraocular lens in an eye, and for insertion into said eye through a small incision, comprising:
a connected pair of V-shaped elements, aligned in the same direction along the length of said haptic.

173. The haptic of claim 172, wherein at least one of said V-shaped elements is rounded.

174. The haptic of claim 172, wherein at least one of said V-shaped elements is square.

175. The haptic of claim 172, further comprising at least one cleat for attachment to an optic.

176. The haptic of claim 172 wherein said cleat is chamfered.

177. The haptic of claim 172 wherein said cleat is fabricated separately and attached to the haptic.

178. The haptic of claim 172, wherein said cleat is tinted.

179. The haptic of claim 172, wherein said haptic comprises two or more cleats.

180. The haptic of claim 172, wherein said arms of said at least one of said “V”-shaped elements is sufficiently flexible to move the other one of said arms of said at least one “V” shaped elements.

181. The haptic of claim 172, wherein said haptic is composed of a material selected from the group consisting of: polyimide, polyetheretherketone, polycarbonate, polymethylpentene, polyphenylsulfone, polymethylmethacrylate (PMMA), polypropylene, polyvinylidene fluoride, polysulfone, and polyethersulfone.

182. The haptic of claim 181, wherein said polyimide is KAPTON.

183. The haptic of claim 181, wherein said haptic is composed of polymethylmethacrylate (PMMA).

184. The haptic of claim 172, wherein said haptic has a modulus of elasticity of about 450,000 psi/inch.

185. The haptic of claim 172, wherein said haptic has a modulus of elasticity of 100,000 to 500,000 psi.

186. The haptic of claim 172, wherein said haptic is less than about 0.01 inches thick.

187. The haptic of claim 172, wherein said haptic is machine-formed.

188. The haptic of claim 172, wherein said haptic is laser cut.

189. The haptic of claim 172, wherein said haptic is molded.

190. The haptic of claim 172, wherein said haptic has a hardness of about 90 to 95 shore M.

191. The haptic of claim 172, wherein said haptic is sized for a particular eye, and wherein one of said legs of said haptic is larger than the space within said particular eye.

192. The haptic of claim 191, wherein the diameter of said haptic is up to about 1 mm greater than that of said particular eye.

193. The haptic of claim 191, wherein the diameter of said haptic is between about 0.3 and 0.6 mm greater than that of said particular eye.

194. The haptic of claim 191, wherein the diameter of said haptic is between about 0.4 and 0.5 mm greater than that of said particular eye.

195. The haptic of claim 172, further comprising an eyelet.

196. A haptic for supporting an intraocular lens in an eye, and for insertion into said eye through a small incision, comprising:
a connected pair of straight or rounded V-shaped elements, aligned in opposite directions along the length of said haptic.

197. The haptic of claim 196, wherein at least one of said V-shaped elements is rounded.

198. The haptic of claim 196, wherein at least one of said V-shaped elements is square.

199. The haptic of claim 196, further comprising at least one cleat for attachment to an optic.

200. The haptic of claim 196, wherein said cleat is chamfered.

201. The haptic of claim 196, wherein said cleat is fabricated separately and attached to the haptic.

202. The haptic of claim 196, wherein said cleat is tinted.

203. The haptic of claim 196, wherein said haptic comprises two or more cleats.

204. The haptic of claim 196, wherein said arms of said at least one of said “V”-shaped elements is sufficiently flexible to move the other one of said arms of said at least one “V” shaped elements.

205. The haptic of claim 196, wherein said haptic is composed of a material selected from the group consisting of: polyimide, polyetheretherketone, polycarbonate, polymethylpentene, polyphenylsulfone, polymethylmethacrylate (PMMA), polypropylene, polyvinylidene fluoride, polysulfone, and polyethersulfone.

206. The haptic of claim 205, wherein said polyimide is KAPTON.

207. The haptic of claim 205, wherein said haptic is composed of polymethylmethacrylate (PMMA).

208. The haptic of claim 196, wherein said haptic has a modulus of elasticity of about 450,000 psi/inch.

209. The haptic of claim 196, wherein said haptic has a modulus of elasticity of 100,000 to 500,000 psi.

210. The haptic of claim 196, wherein said haptic is less than about 0.01 inches thick.

211. The haptic of claim 196, wherein said haptic is machine-formed.

212. The haptic of claim 196, wherein said haptic is laser cut.

213. The haptic of claim 196, wherein said haptic is molded.

214. The haptic of claim 196, wherein said haptic has a hardness of about 90 to 95 shore M.

215. The haptic of claim 196, wherein said haptic is sized for a particular eye, and wherein one of said legs of said haptic is larger than the space within said particular eye.

216. The haptic of claim 215, wherein the diameter of said haptic is up to about 1 mm greater than that of said particular eye.

217. The haptic of claim 215, wherein the diameter of said haptic is between about 0.3 and 0.6 mm greater than that of said particular eye.

218. The haptic of claim 215, wherein the diameter of said haptic is between about 0.4 and 0.5 mm greater than that of said particular eye.

219. The haptic of claim 196, further comprising an eyelet.
